(19) United States
(12) Patent Application Publication
(10) Pub. No.: US 2006/0083743 A1
(43) Pub. Date: Apr. 20, 2006

(54) Method of cancer therapy by in situ production of F5 antibodies

(76) Inventor: Sergiu Calmanovici, Haifa (IL)

Correspondence Address: Martin Moynihan;c/o Anthony Castorina, Suite 207, 2001 Jefferson Davis Highway, Arlington, VA, 22202, US

(21) Appl. No.: 11/143,735

(22) Filed: Jun. 3, 2005

(30) Foreign Application Priority Data
Oct. 19, 2004 (IL) 164683

(51) Int. Cl. A61K 39/395; A61K 9/14

(52) U.S. Cl. 424/155.1; 424/486

(57) ABSTRACT

The invention refers to the production of antibodies named F5 in a cancer carrier and the effect they exert on the development of guest's tumor. F5 may down regulate the proliferation and life of cancer cells regardless the histological nature of the malignancy and the stage of tumor growth. The novel antibody may be raised exclusively in a tumor-carrier guest by challenge with the immunoglobin antigen FIV Abs. This kind of immunization imitates at a certain extent the scheme of classical vaccination with an extern pathogen justifying thus by a semantic derogation the anti-cancer therapeutic vaccination name. The uniqueness of F5 is the result of the adjustment between the conventional antigen FIV to the reactivity in cancer known as premunition. Theoretical considerations and experimental data bring evidence of the efficiency of multi clonal F5 Abs in the treatment of primary or metastatic tumors. The anticancer effect of F5 is only possible as a result of the structural mimicry of the agent with the cancer autocrine growth factors. The blocking of cancer receptors with F5 Abs may produce regression of the primary or metastatic tumors, or arrest of cancer proliferation for indefinite periods of time or, according to circumstances to reduce quantitatively the radio-chemo therapy and the extension of surgical intervention in case of necessity.

### RELATED PATENT APPLICATION

This application claims the benefit of priority of Israel patent application No. 164683, filed on Oct. 19, 2004.

### FIELD OF THE INVENTION

Biological treatment of cancer carrier by production in the body of the patient blocking antibodies (F5) committed to be complementary of autocrine cancer cells growth factors receptors. The adaptive immunological diversity and the intensity of the response are determined by the presence of the tumor and its functional state.

### BACKGROUND OF THE INVENTION

### Natura enim non nisi parendo vincitur

### Francis Bacon, Novum Organum, Partis secundae, Aforism III

The immunological treatment of cancer has developed as an alternative to chemical, surgical or radiological therapy either alone or as a supplement of other more invasive methods. The first attempt to treat breast cancer using the microbial infection of surrounding tissues is traditionally attributed to the American surgeon William Coley, in 1896. However, attempts to change the reactivity of the cancer carrier were recorded even before this date. Nevertheless, the significant impetus for the introduction of biological methods in the medical management of cancer became apparent in the XX century. Contributory to this process were the prestige attributed to vaccination in the post-pasteurian period and, not less, for the last half of that century the vertiginous development of technology and findings of modern immunology. In analogy with the Communicable Diseases, the attempts to treat cancer have led to the development of “anti-cancer therapeutic vaccination” supposed to limit the tumor invasion and the spreading of metastasis by the immune defense. In conformity with the role of disease prevention that is ascribed to vaccination the name therapeutic vaccine undoubtedly represents a contradiction in terms. However, since the term is acknowledged as a synonym for cancer therapy it will be maintained throughout the present work without a semantic correction.

The length of the list of means used so far as cancer therapeutic vaccines may be explained by the fact that none of them has yet proved to be satisfactory, either in individual treatment or in statistical analysis.

The escape mechanisms for survival and proliferation of cancer cells are similar in any tumor, mainly host tolerance and adequate clone selection. However, despite the multitude and diversity of the strategy of cancer therapeutic vaccines, the principle of this Application belongs to other categories.

The antigen F IV used as immunogen in this Application is a biological agent provided with a strong and unique tumoricid effect ⁽²³⁾. The agent is directed against structures that appear in conjunction with the over-expression of the proliferation, either in tumor cells or in tissues, with accelerated physiological growth such as the placenta or the mitosis of the lymphocyte system but without producing any effect such as an immunological response to a specific cellular clone. Unfortunately, FIV could not be used as the ultimate solution in biological cancer therapy despite the strong and selective cytotoxic action that it exerts on cancer cells. The action of the product is limited to the percentage of cancer cells already involved in the mitotic cycle. This percentage does not reach more than a restricted fraction of the tumor mass. Despite the extended histological damage, escape mechanisms prevail and the condition is inevitably lethal due to an increase of the mass of primary tumor and the favoring of metastasis spread.

Considering the existence of spontaneous cancer regression ⁽³⁾, the a priori working hypothesis of this Application is that a successful biological treatment of cancer is possible.

‘Nature to be commanded must be obeyed’. In a realm in which the long list of classical and advanced medical means of surgery, radiotherapy and chemical agents have failed to bring ultimate solutions it is perhaps pertinent to remember the time-honored postulate of Bacon, (translated by himself) and to turn to natural experimental models as a suggestion for cancer cure. As paradoxical as it may sound, the best therapeutic model for the cure of a condition considered as incurable without treatment is the mechanism of spontaneous recovery, if such a situation exists. This malignant disease offers the prospect of the Spontaneous Regression of the cancer bearer's condition although the incidence by which the phenomenon occurs is extremely low varying according to the authors between 1/60,000 to 1/140,000 ^{(10,35,38,54,57,64,70,75).} Behind the statistical data, the frequency or rarity of a phenomenon is not an indication per se of the nature of the biological processes. Important in this case is the recognition of a certain pattern which may be established in both the physiological mechanism of spontaneous healing and in a therapeutic design applicable to cancer. Considering the cases published in the international medical literature as cases confirmed and documented by auxiliary examinations ^{(2,30,78)}, the lists of cancer regression include almost all the principal types of cancer despite the limits imposed by the low absolute numbers. Another parameter of a similar signification is the diversity of the timing of the spontaneous regression of cancer and in the same context, the regression of the metastases, sometimes quite well developed. The spontaneous regression of cancer is the result of a complex process in which more than one component of the humoral or cellular arm of the immune system may participate. This complexity, however, does not contradict the possibility that both of the preceding issues are different expressions of a unique process, namely a conversion of the host's tolerance to cancer. If indeed the spontaneous regression of cancer is the result of a switch in the immune reaction of the host organism to cancer constituents then the curing effort must be directed towards inducing the therapeutic finding under experimental control.

The efforts to achieve this aim have not proved to be very rewarding despite the assistance of modern immunology. From the multitude of methods of active immunization in cancer, two attempts of recent years present special interest because of a formal analogy with the herein Application.

The relationship between the host and the malignant growth may be modified in the desired direction either by raising the reactivity of the immune competent arm of the host or by increasing the antigenic properties of the immunogen. However the distinction is mostly theoretical, since the effects are in practice rather intricate.

The immunological means presently in use attempt in general to overcome the tolerance to the tumor by amplifying the specific immune reactivity to TAA(s). The use of the monoclonal antibody technique has made possible the raising of antibodies against certain defined epitopes of cancer antigen and thus enabled disposing of a population of homogenous idiotypes^{(3,39,41)}

The anti-idiotypes antibodies ^{(5,6,7)}, also called ‘surrogate of antigens’^{(43,51)} may reproduce in their variable portion the epitopes of the initial antigen ^{(36,37,45,55)} with a quite close similarity. Since the antigens may sometimes be more immunogenic than the original epitopes, great medical hopes have been raised by the bioengineering of future cancer vaccines using this method ^{(46,47,58,60)} or using close xenoantigens ^{(42,49)}. Unfortunately, the remarkable achievements in the technique and knowledge of cancer immunology are not paralleled by a similar progress in the biological treatment of this condition. One of the impediments of the prolonged administration of monoclonal anti-idiotypes is the impossibility of continuing the treatment because of adverse anti-species reactions, usually anti-mouse. Secondly, the tumor escape mechanism uses the Darwinian phenomenon of clonal selection to raise clonal populations resistant to the vaccine reaction produced in the host. The necessity for vaccination with new monoclonal anti idiotypes thus opens a course between the tumor aggression and the defense means of the organism which sooner or later ends with the surrender of the host ^{(8,9,25,50)}.

The immunogenic effect of tumors, of any malignant tumors, occurs early in the development of the growth and is not subject of doubt. The medical problem is not the real lack of an immunologic reaction but the lack of efficiency or in other words the neutralization of the defense reaction.

Any attempt of biological treatment of cancer is centered on the breaking of the immunological tolerance. On one hand this includes, procedures that may raise the defense mechanisms used against the TAA(s), as above. On the other hand, there have recently been attempts to break non-specifically the tolerance to TAA(s) and producing on experimental models cross immune reactions by utilizing xenogen antigens of animal or human origin.

However, apart a temporary effect, the active immunization based on the clonal kinship of the antigens has not yet been successful in circumventing the pitfall of immunological tolerance to cancer. Speculatively, it looks as if the clonal selection being a derivate of cell proliferation of the metazoan organism also includes the propensity for malignant transformation as an inherent expression of the cellular evolution.

The humor immunological reaction against cancer that forms the object of this Patent Application is based on a different approach. The antigen is, in this case, related to general cellular functions perpetuated in cancer e.g. the cellular division. As a basic cellular function it precedes in phylogeny by about six hundred millions years ^{(4,48)} the mechanisms involved in the clonal differentiation of vertebrates.

A suitable antigen for serving as cancer immunogen was found in this Application by replacing the usual immunochemical criteria of selection by the functional properties of the molecular structures. The procedure used in the present has led to the elicitation of Cancer Electromyographic Evoked Pattern (CEEP) as the effect of a Cancer Humoral Factor (CHF) on the Neuro Muscular Junction (NMJ)) of the frog ^{(14, 15 and 16)}. The novel method of antigen separation enabled the raising of the anticancer sera named FII, provided with novel anti-cancer properties ⁽¹⁷⁾.

One special immunological feature of the variable domain of this population of antibodies is the property of the novel serum to behave functionally as either mono or multi clonal antibodies (FII Abs). This property enabled the use of antibodies as an antigen of a special type, and preparation of a positive copy of antigenic determinants, defined as F III Abs. Moreover, FIII Ab or its F(ab)′ fraction has served as antigen for a novel FIV serum, actually the anti-anti-idiotype antibodies of FII in which the mono or multi clonal character of the original antibodies-antigen is further conveyed and amplified.

However, the physiological functions of FII, FIII and FIV Abs do not reflect the clonal expression of the cells involved in the Ab production, neither in allogen individuals and evidently nor in distant species, genetically completely different. The integration of these novel antibodies in the complexity of the immune system should be carried out differently from that of the antibodies raised by conventional antigens.

The multifunctional aspects of both agents, FIII and FIV are detailed in the Specifications of the Patent Applications ^{(22, 23)}. In the respective Applications it is stressed that both factors are cytotoxic for cancer cells to an extent not yet attained with any other biological agent. However, despite the apparently temporary beneficial effects of this feature, this cancer citotoxicity cannot provide a medical treatment solution. Both factors induce regressive changes that lead eventually to the damage and destruction of a percentage of the cancer cell population. Both agents arrest the mitotic cycle, initiate serious metabolic changes in cancer cells and may induce cellular death by means of apoptosis. The percentage of the cancer cells affected is initially approximately the same in the treatment with either of the two agents. In the treatment with FIII Abs the tumor and lymphatic ganglions undergo a magmatic transformation in parallel with the continuous tumor growth. (FIG. 1-a,b,c,d,e,f,g.) After FIV administration, big cystic cavities appear in the bulk of the tumor, filled with an opalescent liquid, dispersed or collected in a system of cysts. However, in both cases, the course of tumor development is not stopped and the animal's survival is not prolonged.

The restriction imposed on F III and F IV in their use as therapeutic factors is a direct reflection of the proliferation characteristics of the tumor. In both cases the citotoxic effect is related absolutely to the growth of the tumor although the mechanisms of action of the two factors are different.

The restrictions imposed on these agents in the attempts to be used as anti-cancer agents result from the biological condition of the tumor cells at the time of the treatment. F111 blocks the internalization of Growth Factors or, in the case of FIV, restrains the supply of GF-like ligands, but only in cells in mitotic cycle. The cell proliferation in cancer is not synchronic but is carried out in successive fractions of growth of malignant tumors. The essential nutrient contained in the GF or GF-like do not enter the cell by membrane diffusion but by receptor binding and cell internalization, hence this pattern of growth is the most appropriate to ensure in a defined period of time a maximal concentration of essential factors in the cell microenvironment.

The asynchronous proliferation of contingents of tumor cells favors maximally the nutrition supply of at least a percentage of the cancer cells involved in the process, of proliferation. Moreover, one of the most important tumor escape mechanisms is based on the biological protection against foreign, agents conferred either by the status of ‘dormant cells’ or by maintaining the tumor bulk in a ‘quiescent’ state. The tumor cells, although very resistant in the rest periods are exceedingly sensitive to therapeutics in some intermediary phases, although one has to notice that these only may last for a very short time. The propaedeutical consequences of a rational strategy of the tumor escape mechanism is that the biologic agent should cover in time or slightly precede the active changes of the cellular cycle (FIG. 2c-1, 2 and 3)

The raising of a map of proliferation of the whole tumor is not possible. The ‘internal clock’ that determines the level of activity of tumor fractions is not under control. The application of the properties of FIII and FIV in a comprehensive treatment of cancer is only possible by finding a medical strategy in which the potential of these agents is integrated in the biological behavior of the whole tumor as shown in the following sections.

### SUMMARY OF THE INVENTION

The main points considered in the elaboration of this invention are (i) the acknowledgement of the immunological tolerance to cancer as the major deficiency of the host organism, (ii) the failure of the attempts to raise an anti-cancer immunological reaction and finally (iii) the separation of a category of antigens exempted of the dependence of TAA(s) on clonal selection. It was possible to achieve this last point by an innovatory neuro-physiologic method by which the selection of antigens was carried out according a functional criterion ^{(14,15 and 16)}.

The achievement enabled the production of a series of sense and anti sense polyclonal antibodies described in detail in other Applications for Patent. (Calmanovici: Preparation of Idiotypic Antibodies to Cancer Humoral Factor—CHF, Production of Antigenic Determinants of Cancer Humoral Factor by Anti-Idiotype Antibodies—FIII and Production of Competitive antibodies to Growth-Cholinergic Receptors—FIV Abs). Finally, the use of F IV Abs or rather of the F(ab)′ fragment as the antigen of active immunization of cancer-carrier organism led to the production of the F5 Abs that display both effects cholinergic and involvement in the cancer proliferation. (fig 2, a, b and c).

The mimicry of F5 to auto and merocrine transformed Growth Factors enables its binding on cancer cell receptors and the inducing of a therapeutic effect by impairing the metabolic ways of cancer cells.

According the conventional definition of the process of “vaccination”, in the absence of cancer epitopes in the antigen, the production of F5Ab with anti-proliferative effect would not be possible. However, a different course is induced in the case of cross-immunity of FIV with the wild epitopes of cancer cell receptors. As it is the case in premunition, the cancer carrier reacts to the challenge with the FIV antigen by production of a type of surrogate of ligand of Growth Factor receptor that is actually the F5 Ab.

Due to the antisense structure of the immunoglobulin-like antigen the immunological response is enough vigourous for yielding a therapeutic effect in situ. Moreover, by use of adequate adjuvants the production of F5 may be unusually augmented for hundreds of times inducing thus a cataclysmic destruction of the tumor tissue in few hours, as shown in the FIG. 20 a,b,c,d and e.

### BRIEF DESCRIPTION OF THE DRAWINGS

FIG. 1-a: Abdominal Lymphatic ganglia.

FIG. 1-b: Primary Tumor of Rabbit (case 1889) inoculated with VX2 tumor two month ago and treated by injection of FIII. Fixation in Formalin 4%.

FIG. 1-c: Another case with an identical history and huge development of the Primary tumor, not yet fixed in Formalin.

FIGS. 1-d, 1-e and 1-f: the microscopic aspect (H/E) shows the generalized cellular destruction and cellular rests of the magmatic noticed macroscopic.

FIG. 1-g: TUNEL reaction of kidney. Elimination by urinary tubules of apoptotic rests of the cellular mass.

FIG. 2-a1: Preparation 872, Snail (slug), H/E staining. Control.

FIGS. 2-a2 and 2-a3: Preparation 872, Vital Immunohistochemical Staining with F5.

FIGS. 2-b1 and 2-b2: Presynaptic nerve network of the electric organ of Torpedo Electricus. Immunohistochemical Staining with FV.

FIGS. 2-c1, 2-c2, and 2-c3: Vital immune histochemical staining of Hepatoma in rat with F5. The procedure enables the localization of sites of action of the agent in vivo.

FIGS. 3-a, 3-b, 3-c, 3-d, 3-e, 3-f and: 3-g: Reproduction of the Poster “Plasma Cells In Squamous Lung Carcinoma; Relation With Premuntion.” In previous meetings of this Society (1, 2) and of Neurological Medical Association a novel antibody named FIII has been presented. The FIII agent has affinity for cholinergic and growth factor receptors and may be used also as the primary antibody of an immunohistochemical staining (IHCS) reaction. The production of, FIII is based on the separation of the cancer antigenic determinants (named F1 or CHF) by a physical method of separation of the antigen (3). The further generations of antibodies reproduce in their variable domains the molecular structure of the original antigen and behave functionally as ligands of growth factor receptors of cancer cells. This working hypothesis was confirmed by IHCS tests performed on human and experimental cancers. In addition to this positive staining in certain cases of squamous carcinoma of the lung appear also plasma cells positively stained by the primary antibody of this reaction. There were also noticeable agglomerations of positively stained formations of white cells.

FIII as the primary antibody located in the transmembrane receptor sites of cancer cells may explain the immunohistochemical staining of cancer tissue. This location, however, does not explain the appearance of specific staining affinity of plasma cells in the cancer tissue or peritumoral compressed normal tissue. This effect may be due to an immunological response of some groups of B cells that react to the challenge of cancer products with a reduced or aborted production of antibodies or intermediary Ig-like adhesion factors. Most of the plasma cells did not appear to be in a secretory or releasing state (blockage of release of antibodies). It is possible however that some of the adhesion factors reached the general circulation by breaking of the old cells and had been absorbed by the circulating white cells.

The term PREMUNITION does; not indicate an immunological sequence of events but means a special state of immunity that is dependent directly on the presence and/or effect of the pathogenic agent on the host organism. A reasonable candidate for such an agent could be the antibody induced by shed antigens of the tumor, especially in selected cases of cholinergic antigens like the pulmonary tumors. Nevertheless, the expression premonition is considered in modern immunology as an obsolete theoretical concept; without objective foundation. The detection of staining affinity properties of the plasma cells was made by chance and was due to fortuitous staining with FIII of a tumor item. In a certain way, that is an embodiment with an external agent of Jerne's theory of producing large networks of anti-antibodies by challenge with the variable domains of suitable idiotypes.

The staining of the plasma cells by the artifactual antibody FIII confirms thus the reactivity of this ligand to the premunition of susceptible B

The study presents morphological changes and Immunohistochemical Staining (IHCS) in a case of Squamous Cell Carcinoma in human lung and the blood cells lymphocytes of the regional peri tumoral tissues.

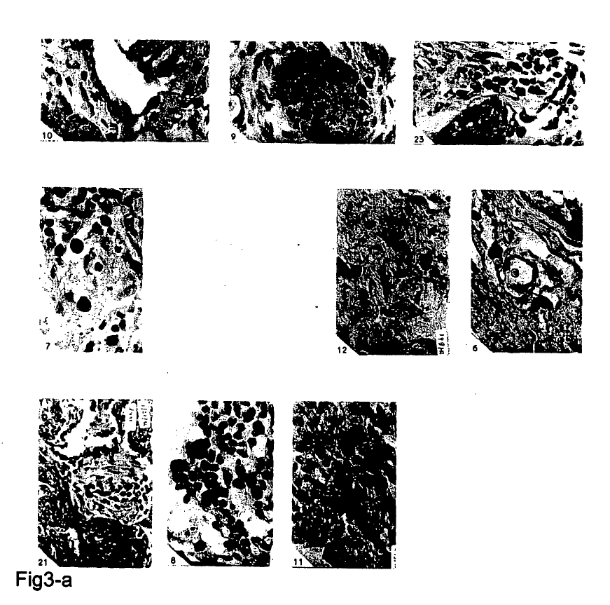

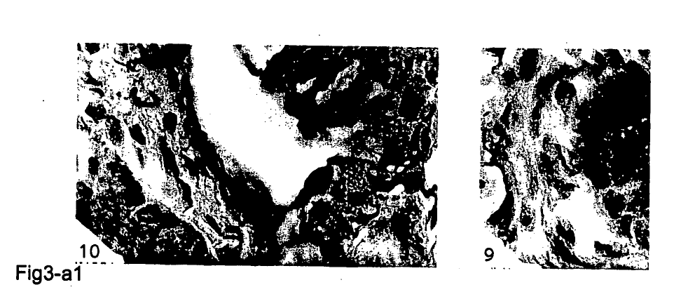

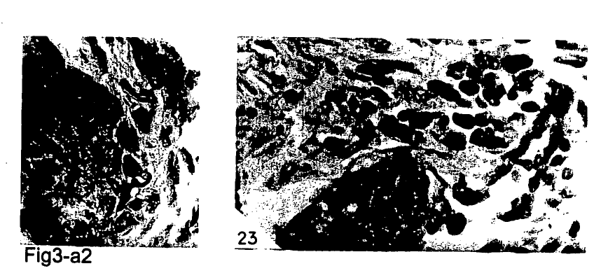

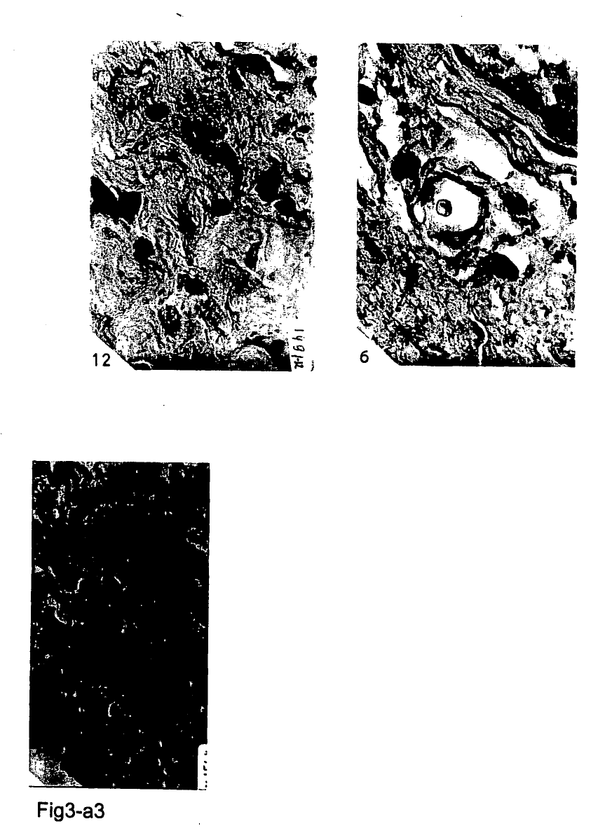

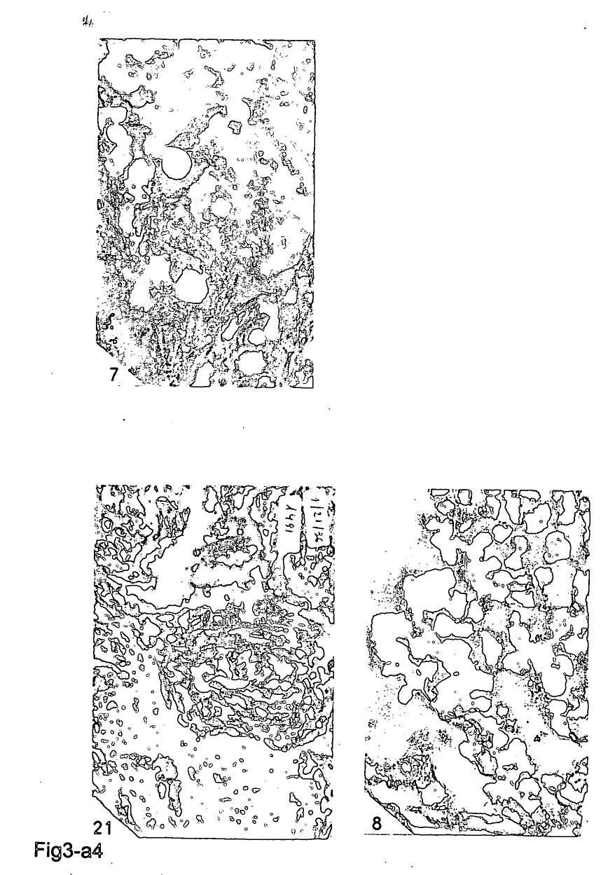

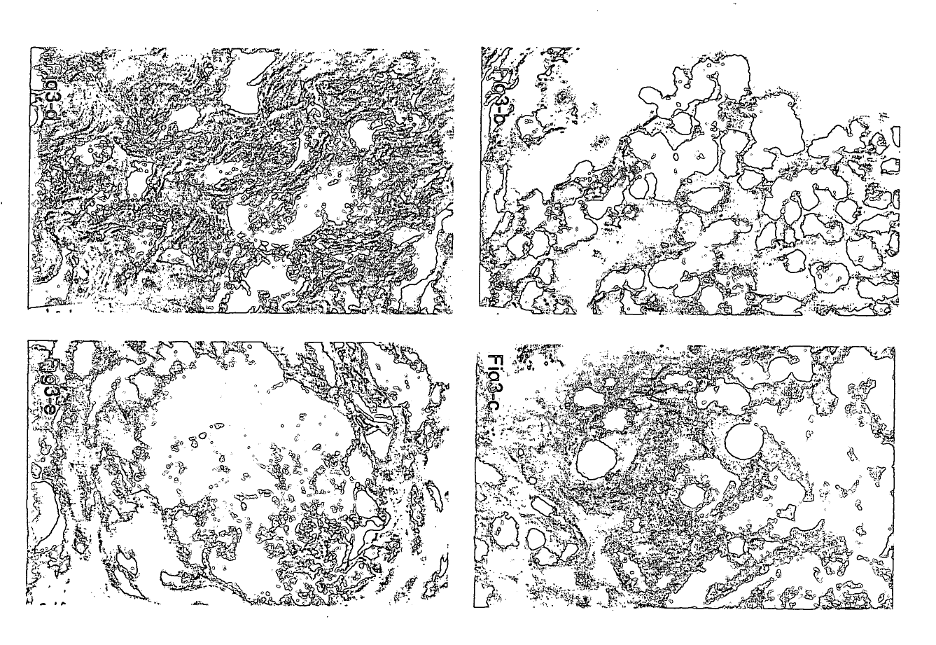

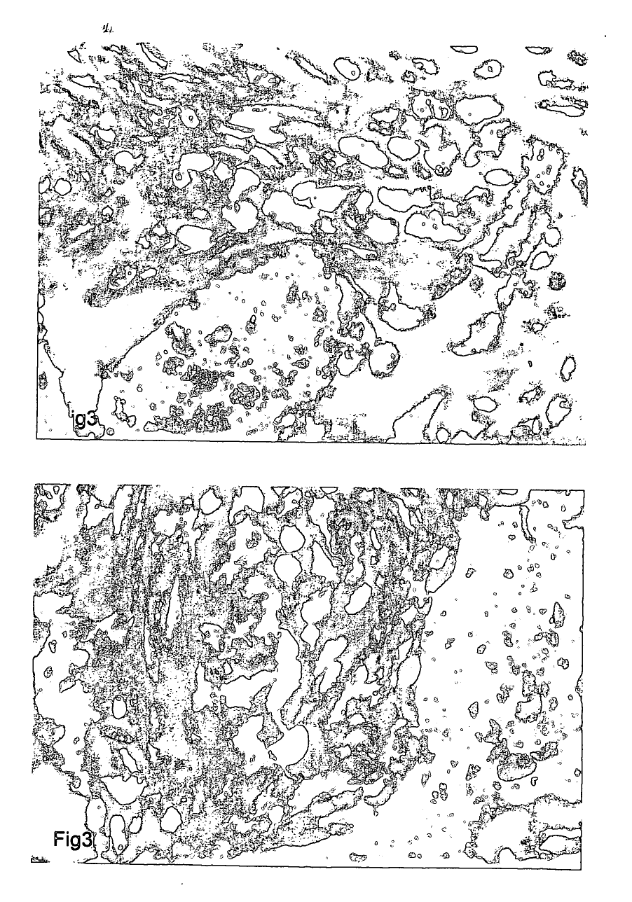

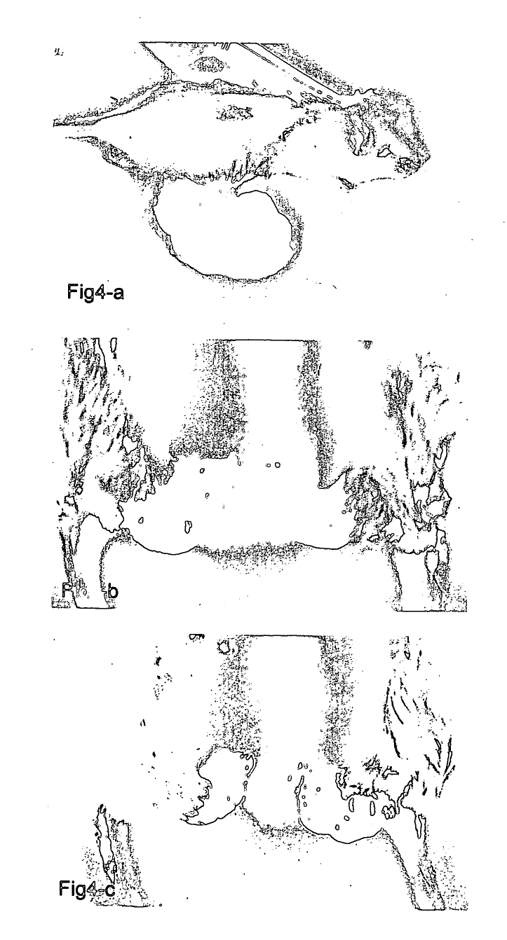

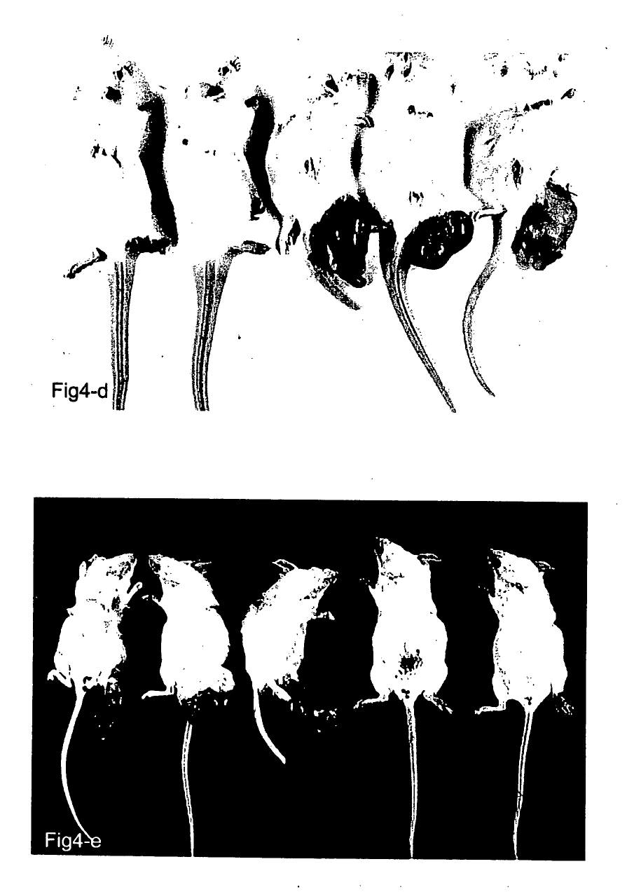

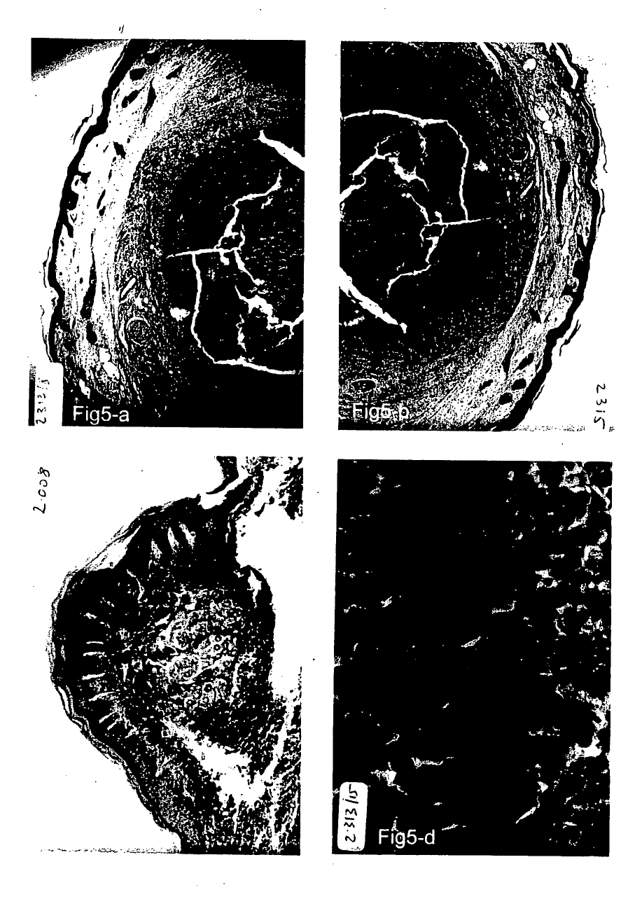

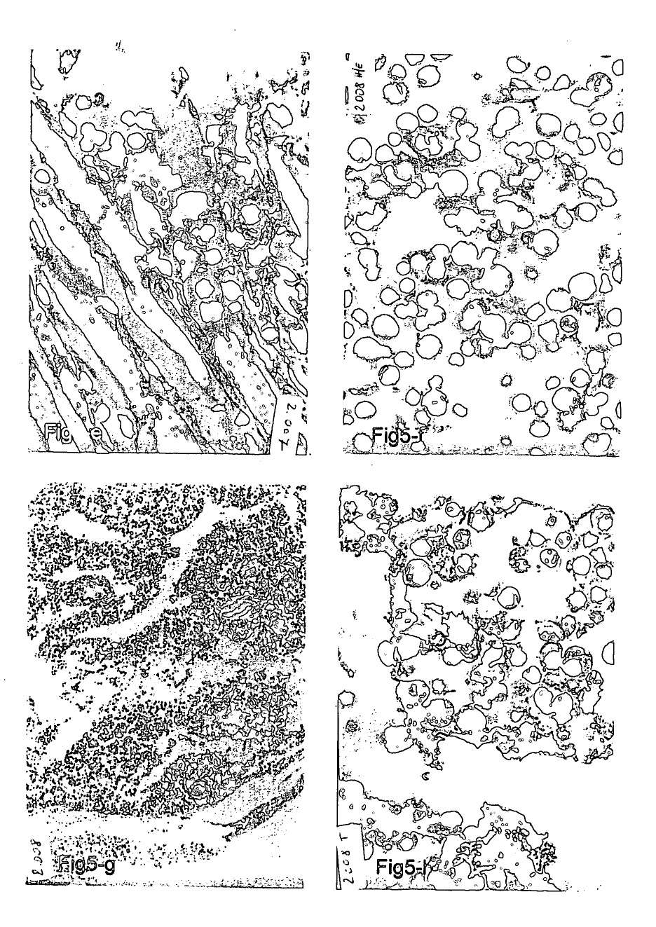

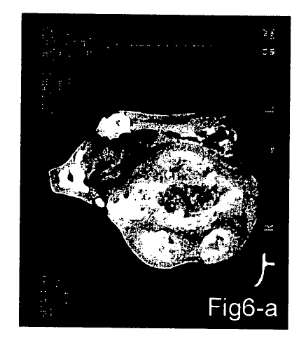

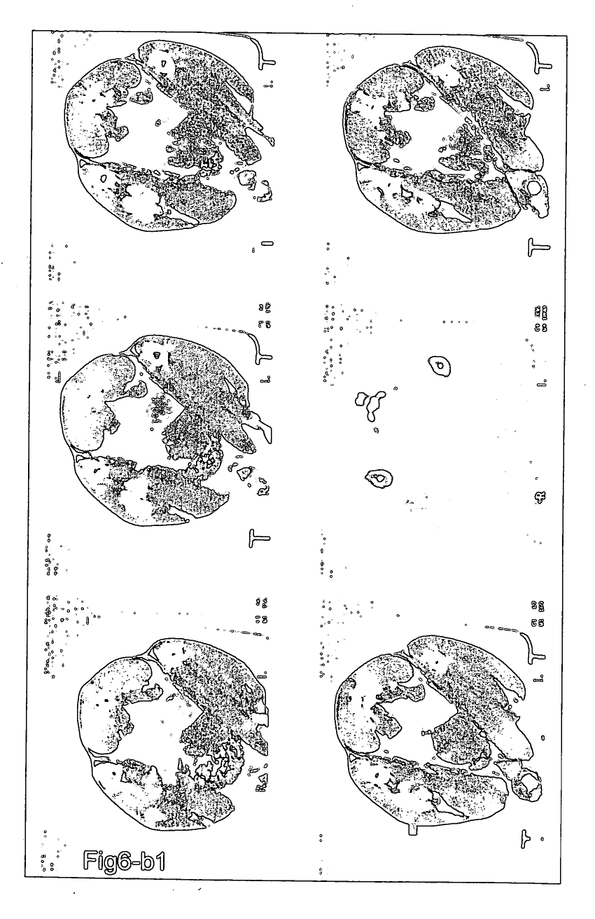

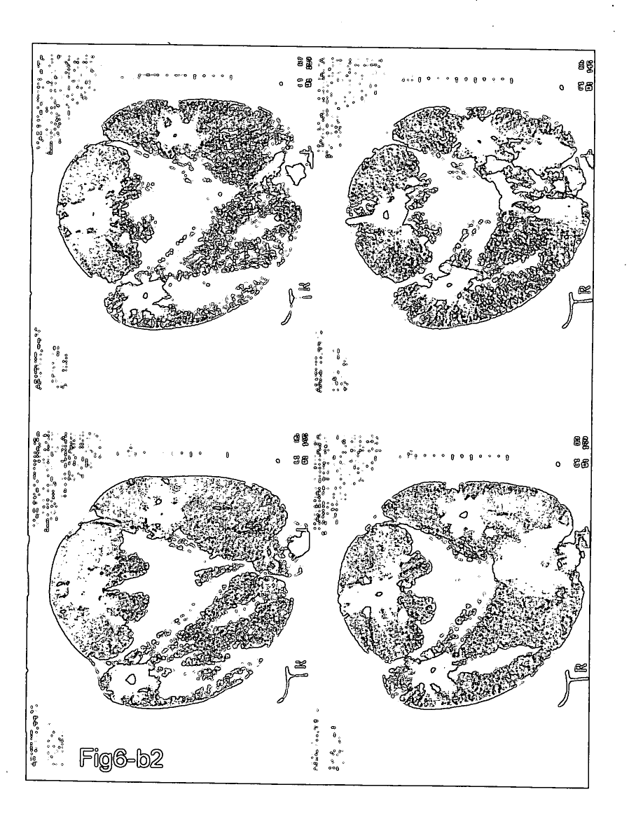

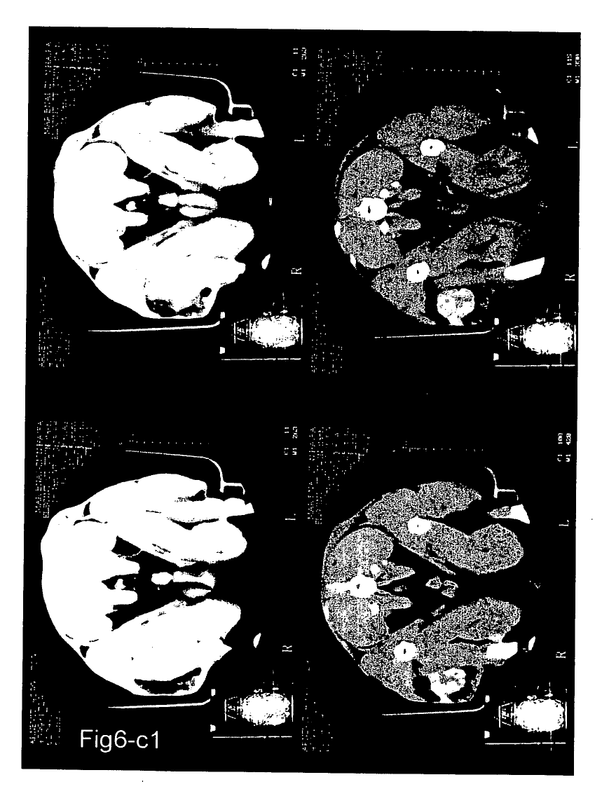

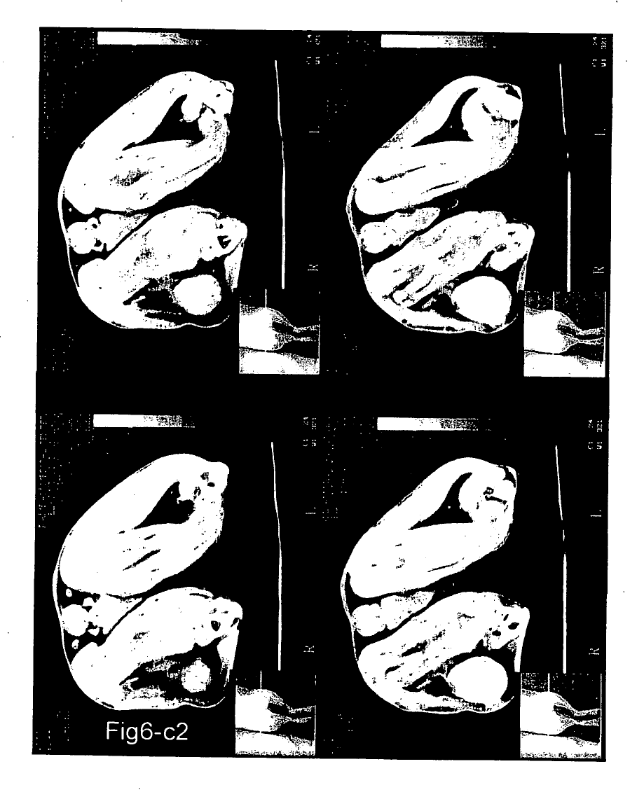

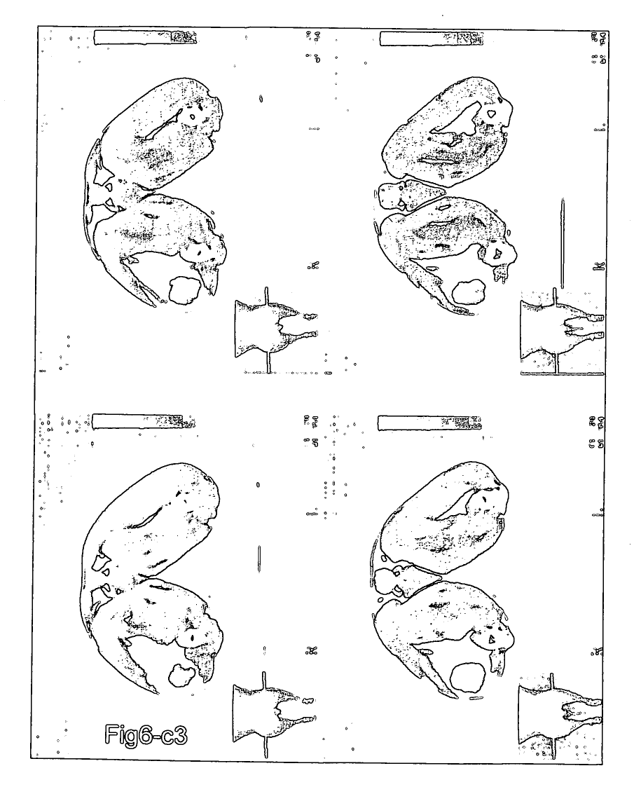

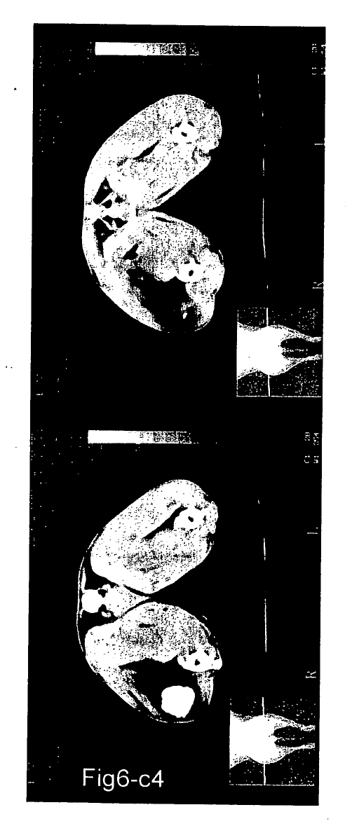

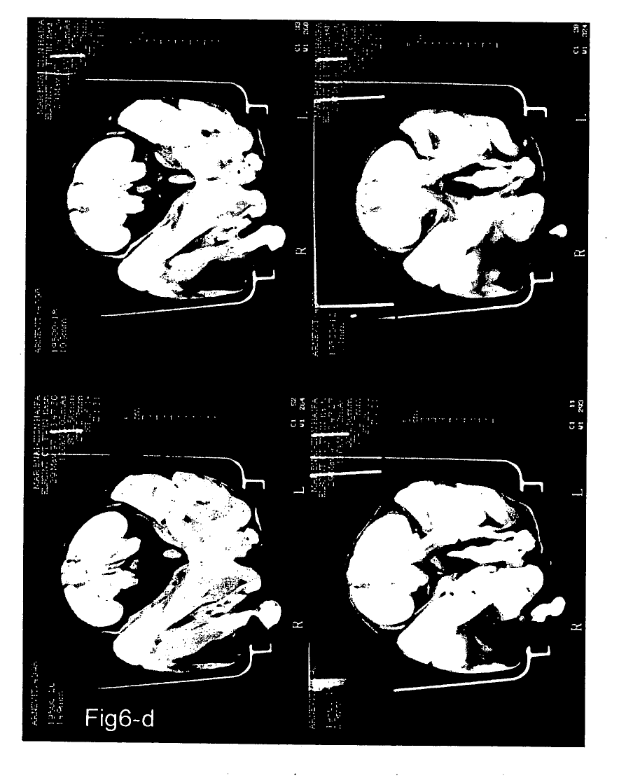

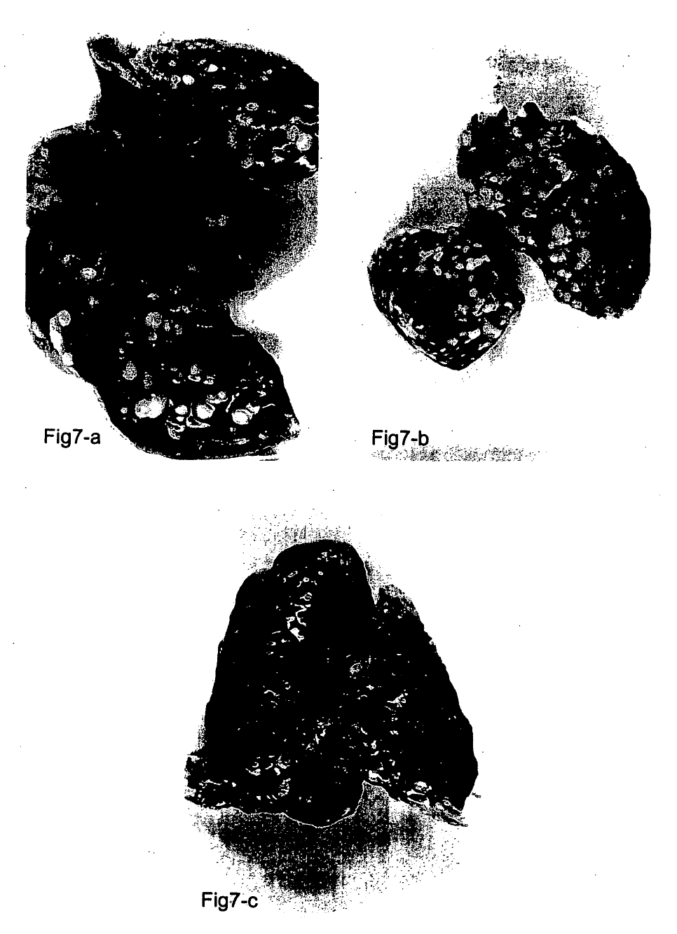

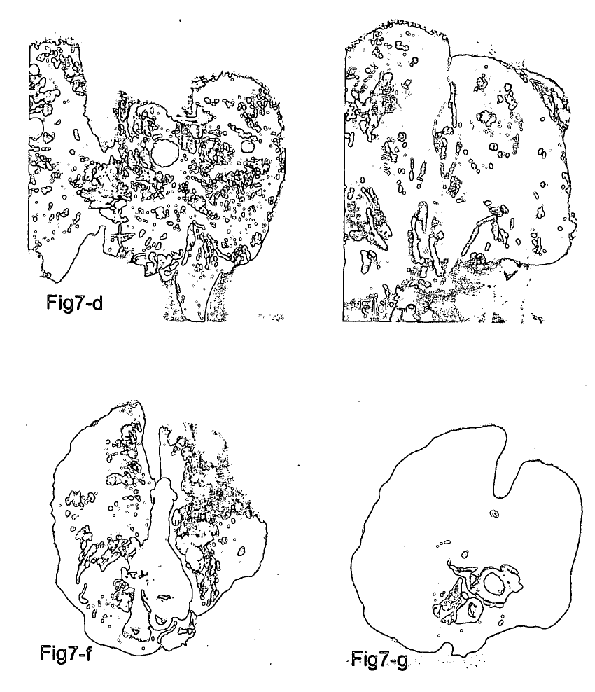

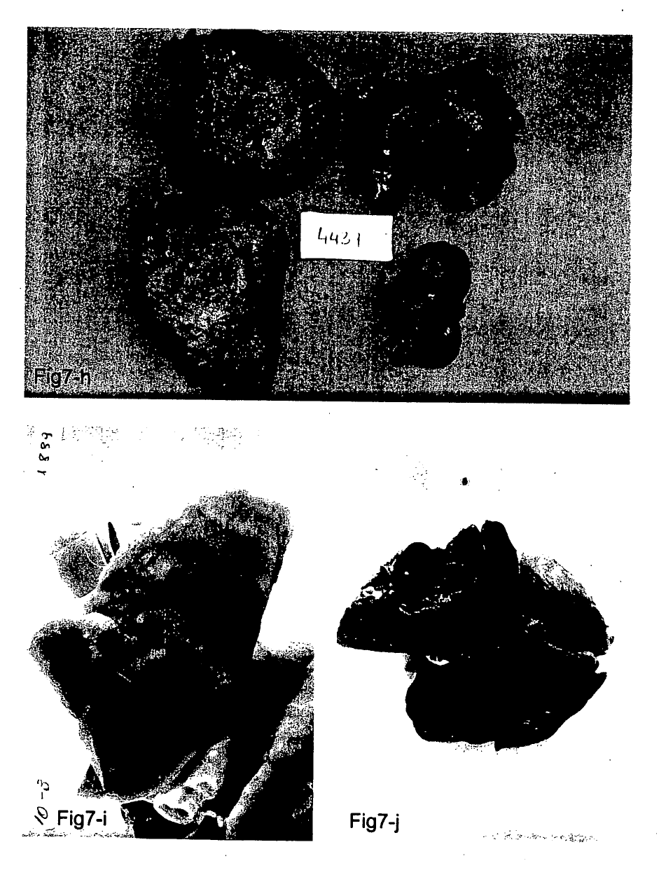

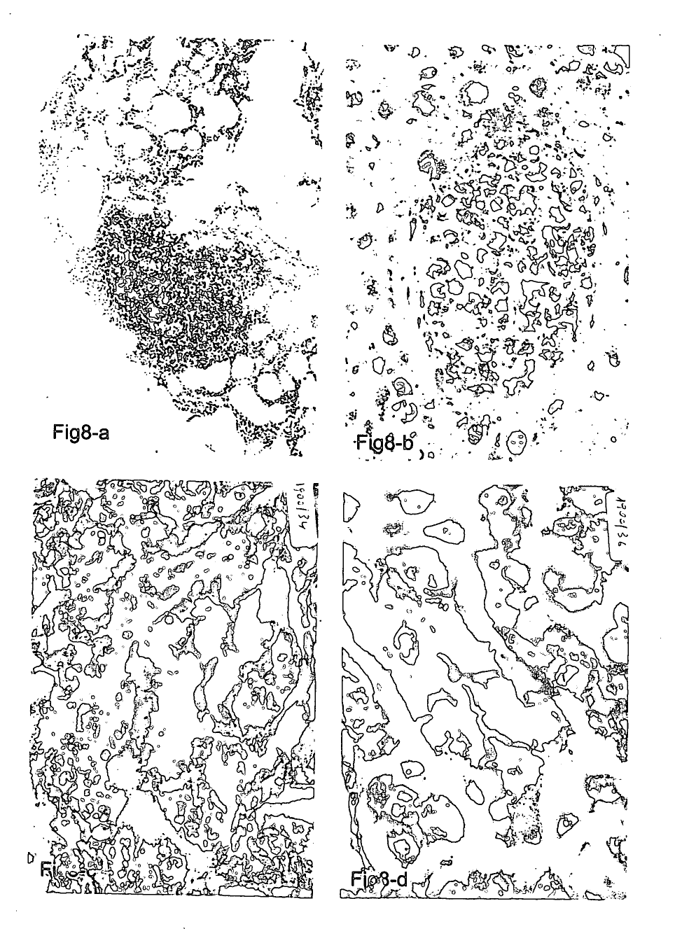

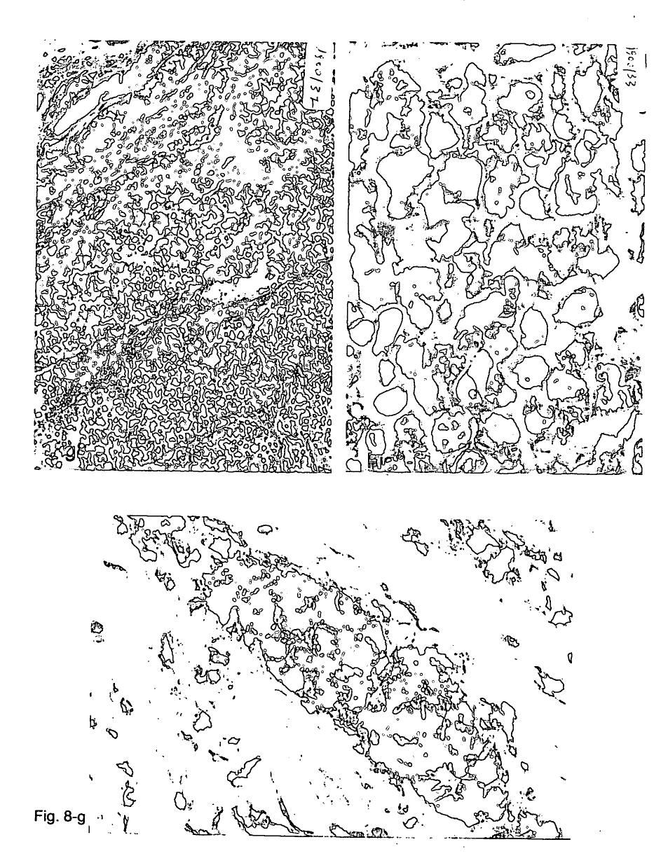

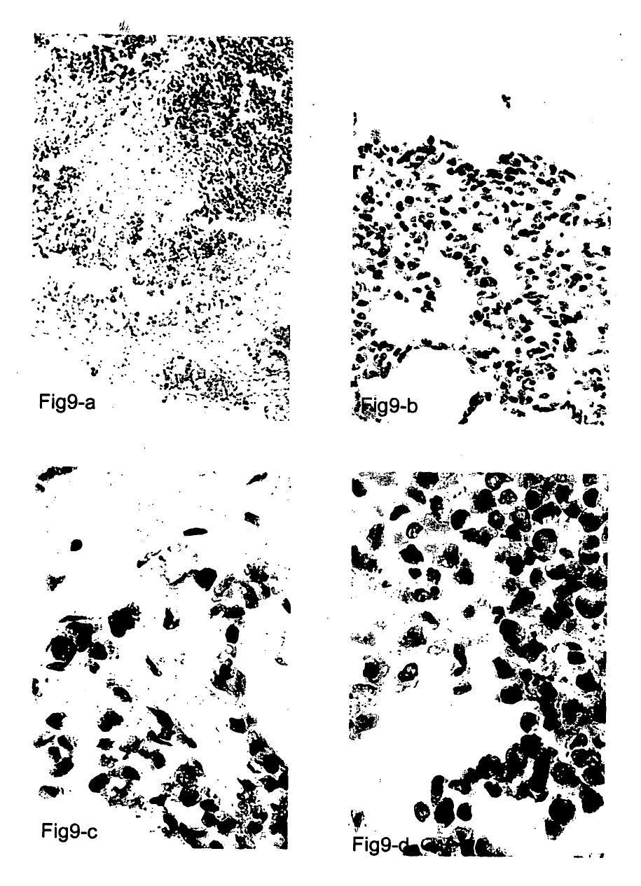

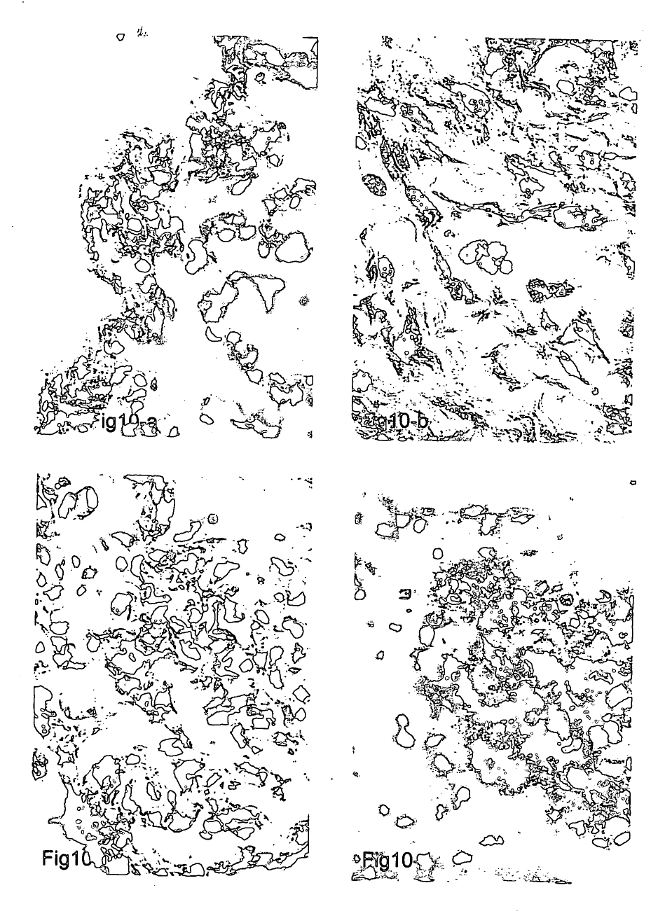

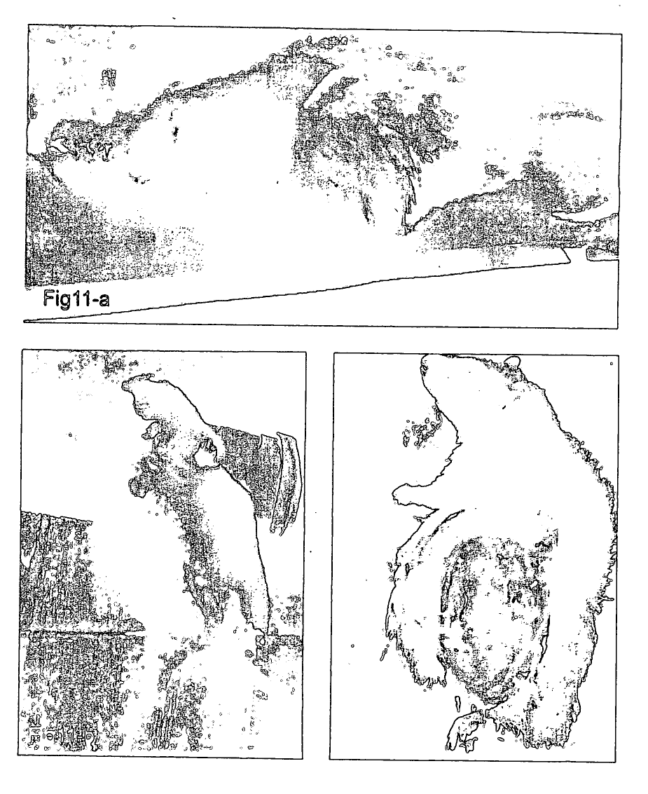

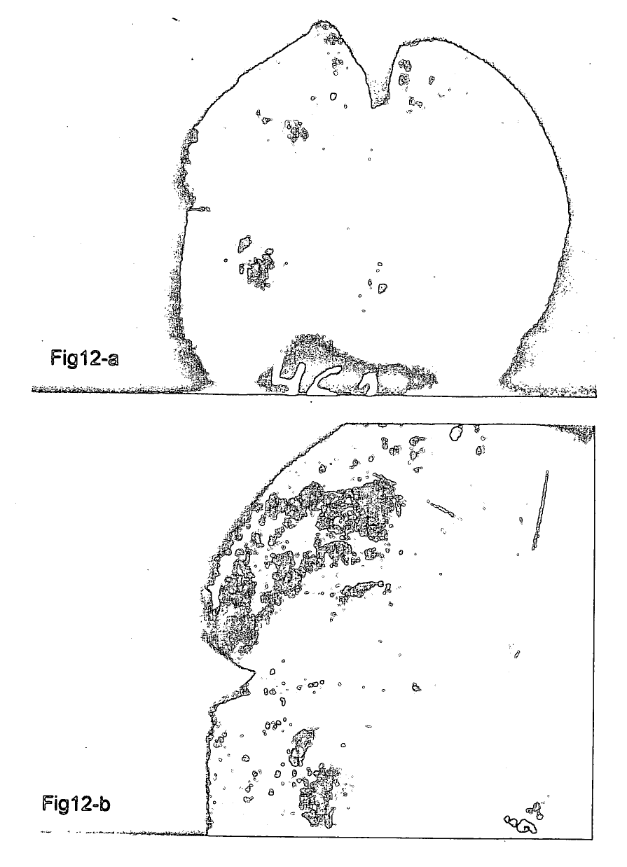

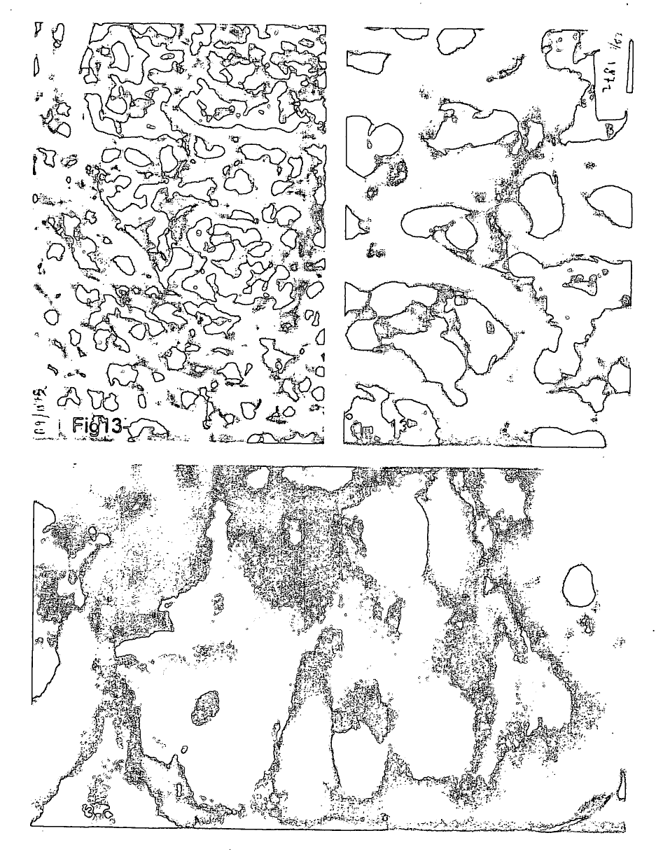

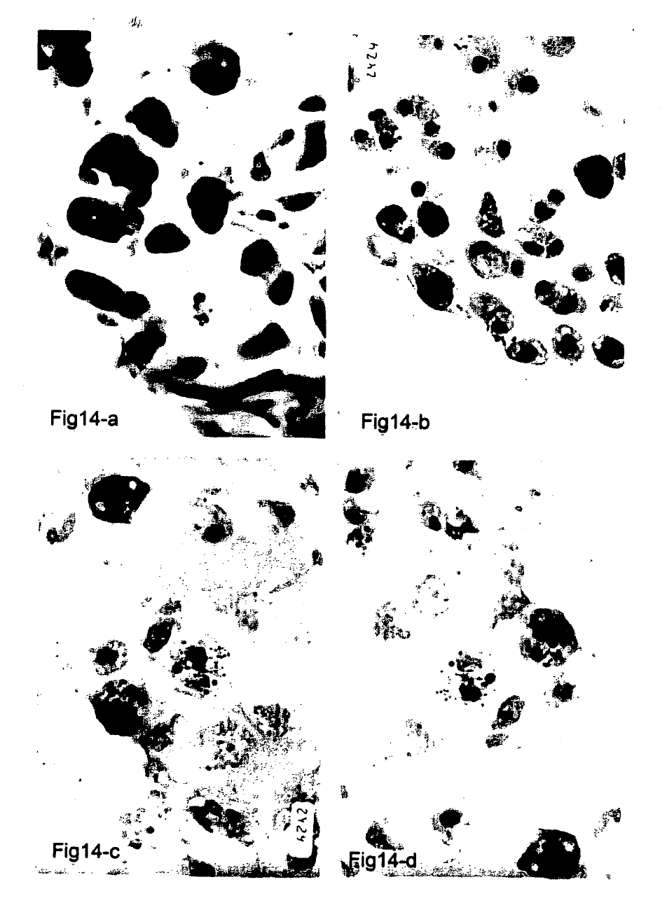

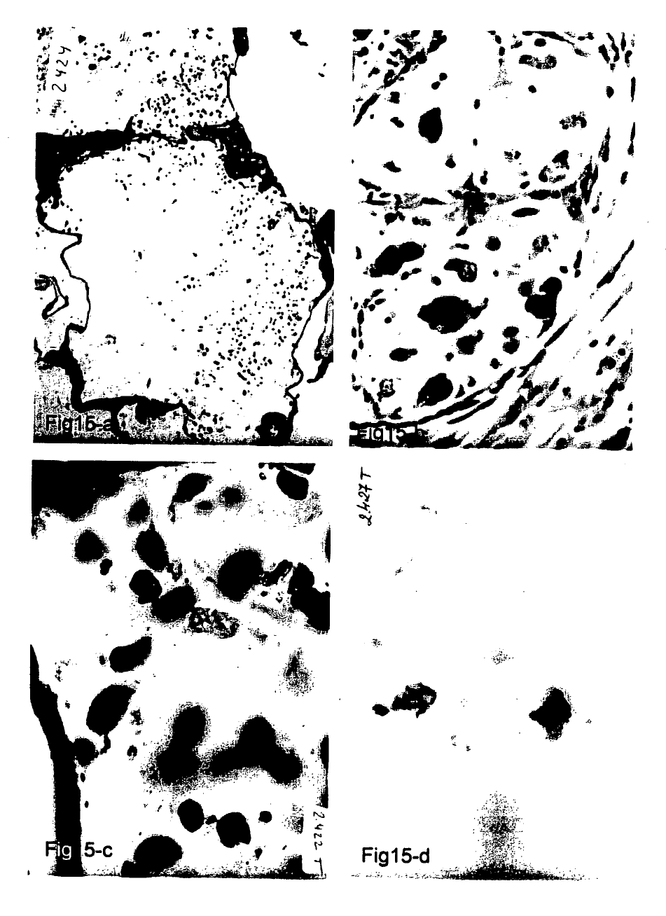

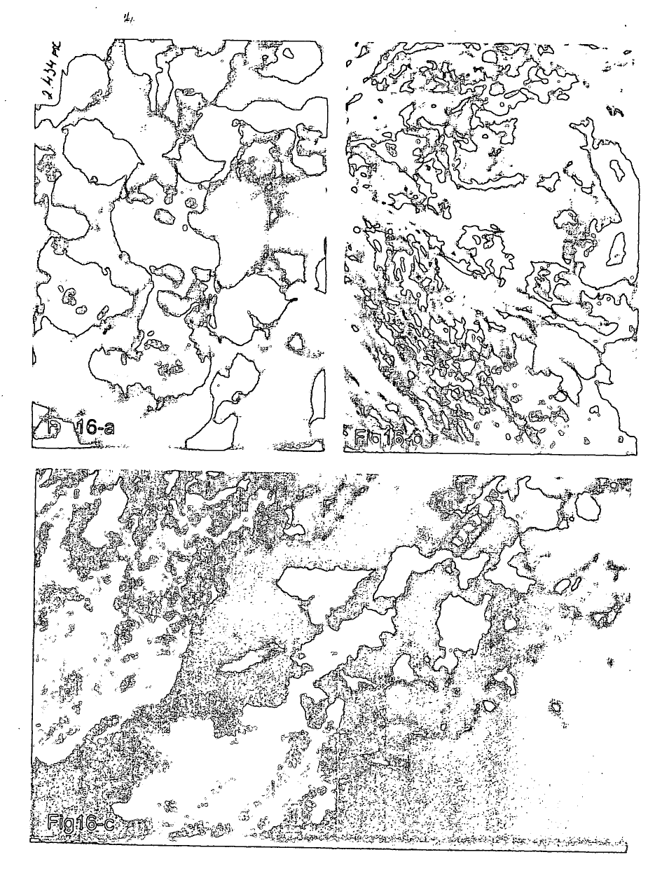

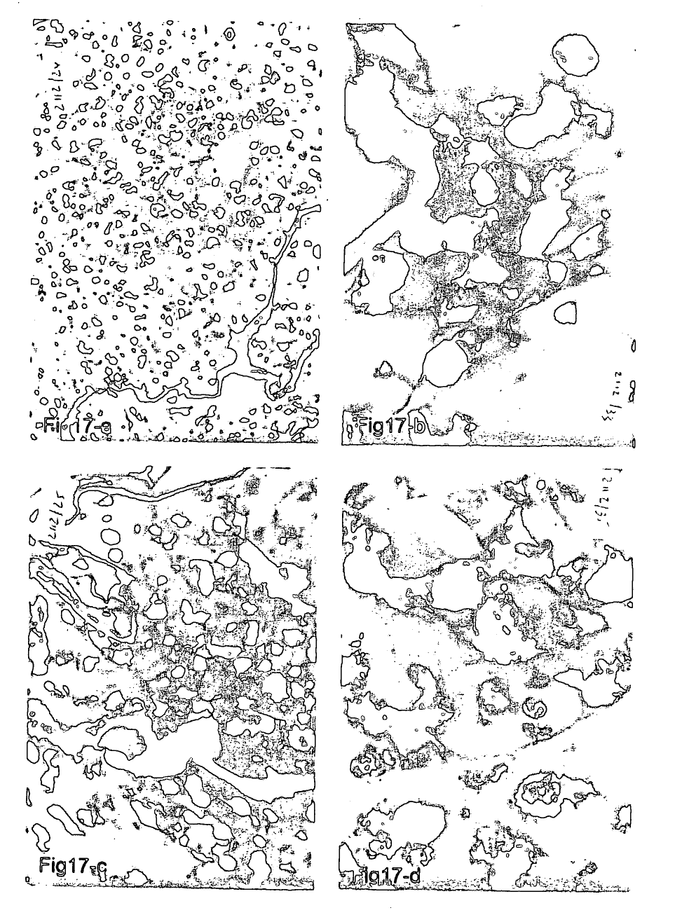

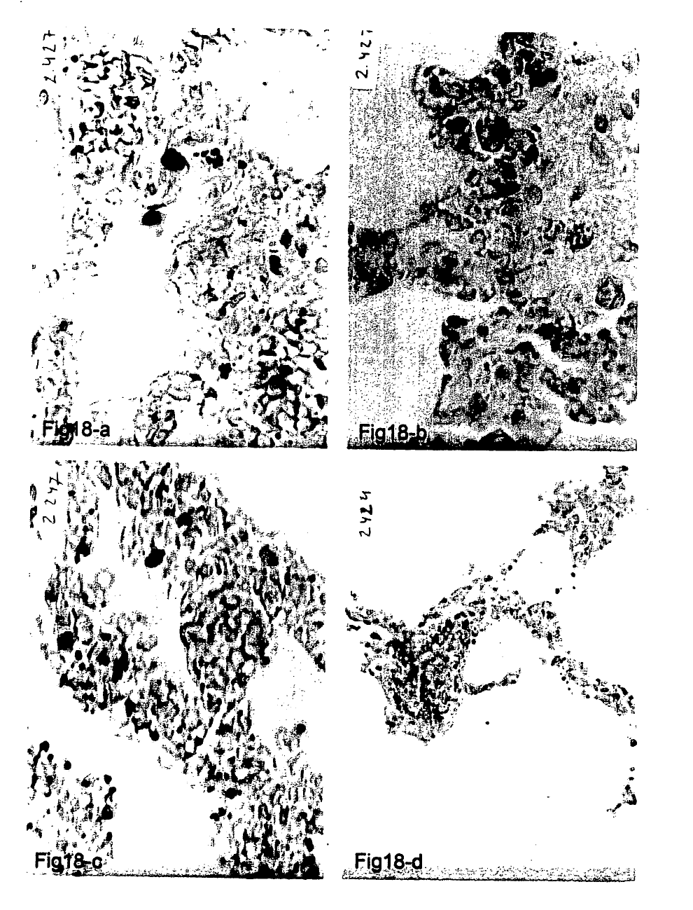

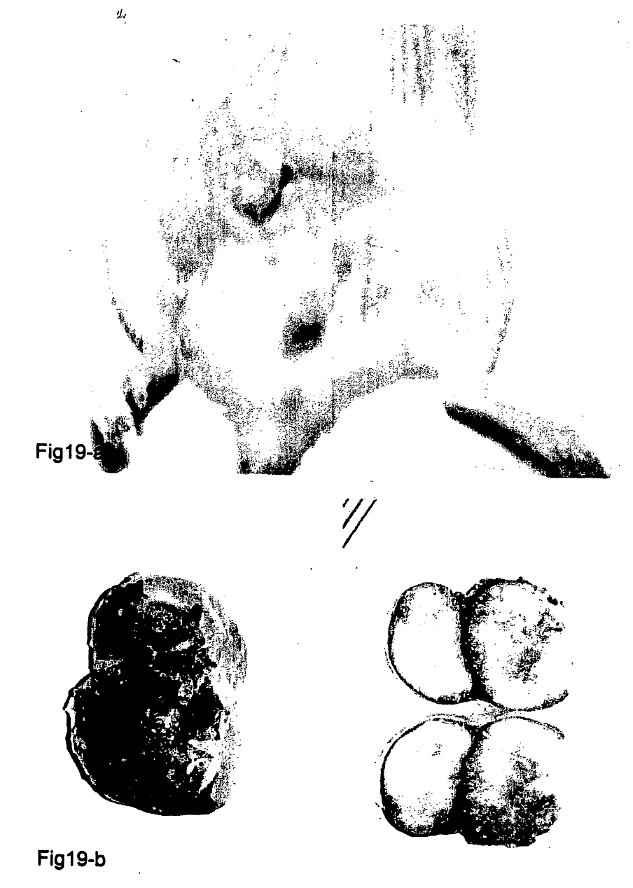

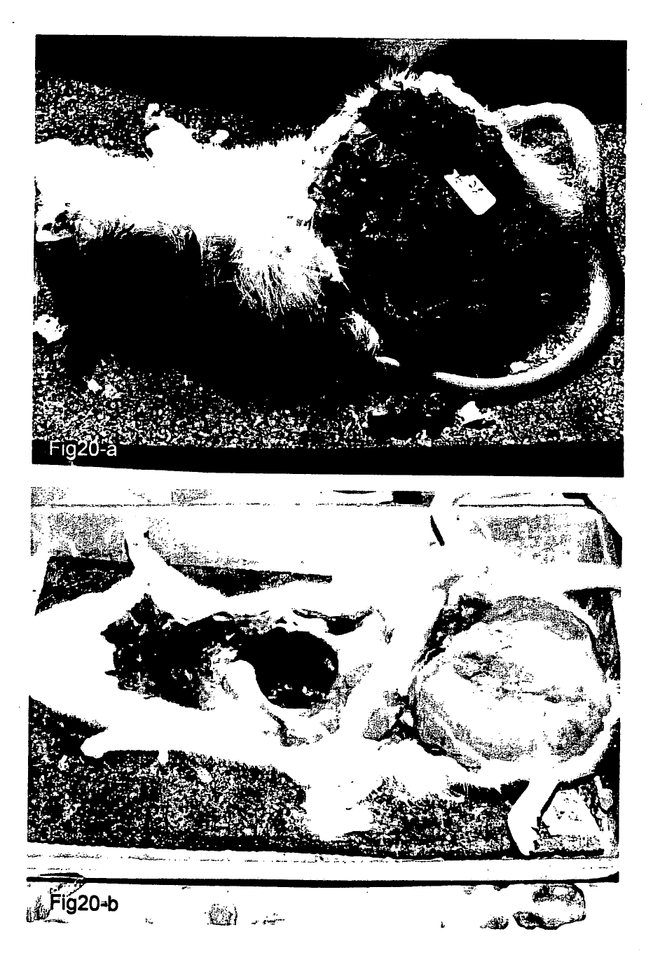

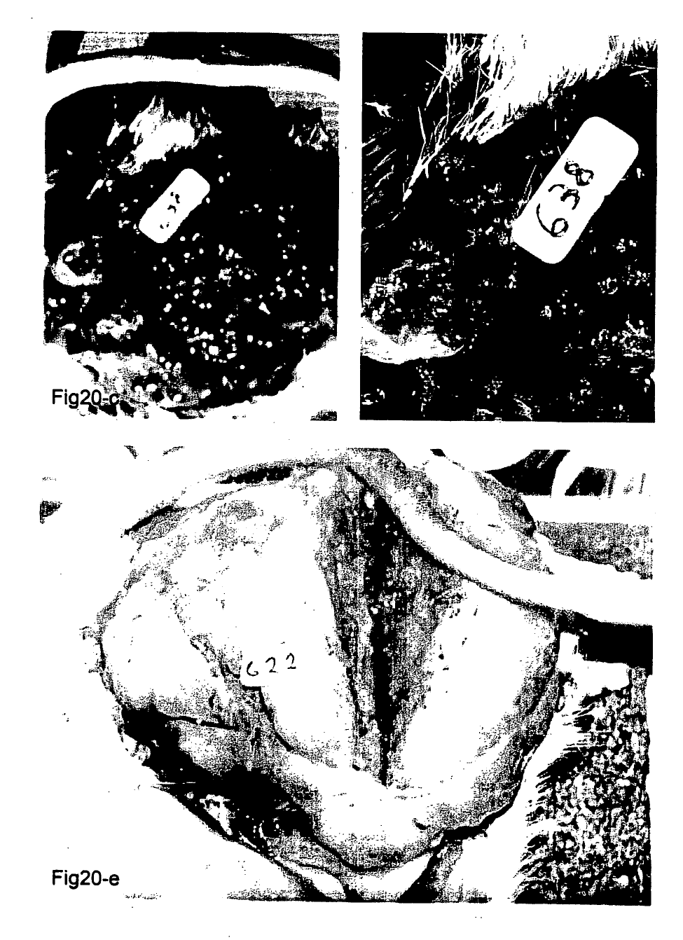

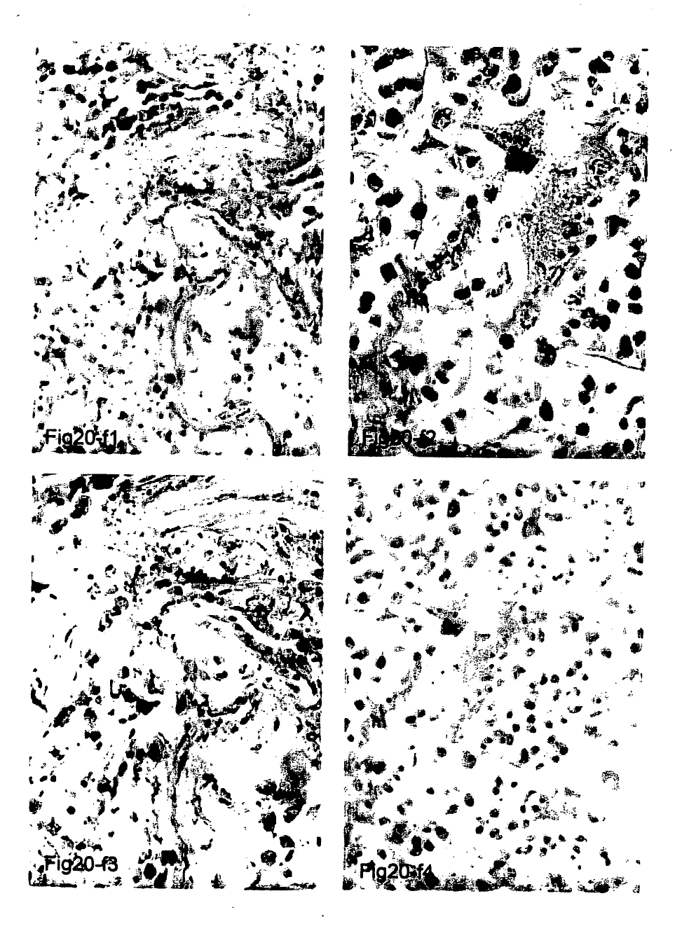

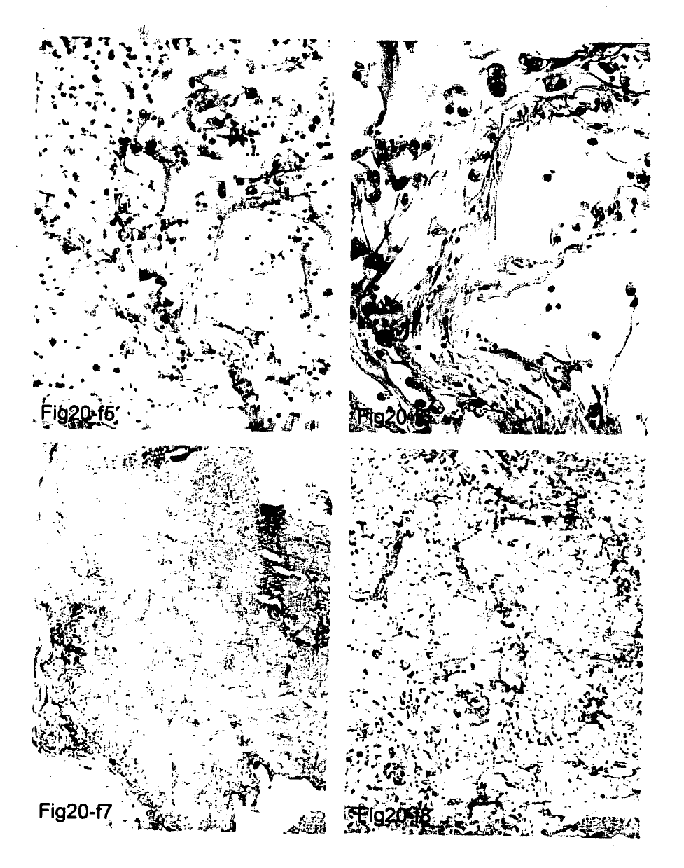

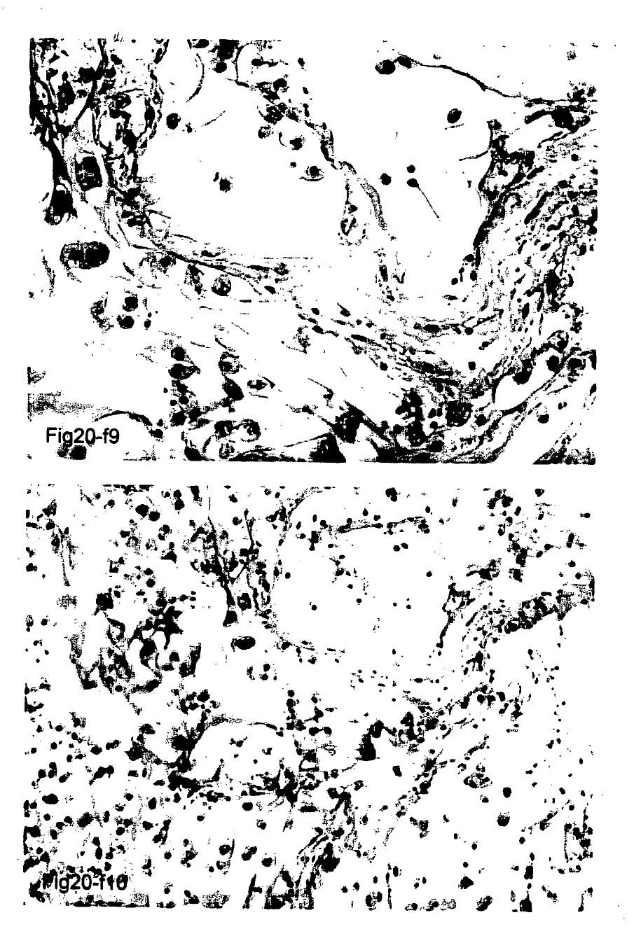

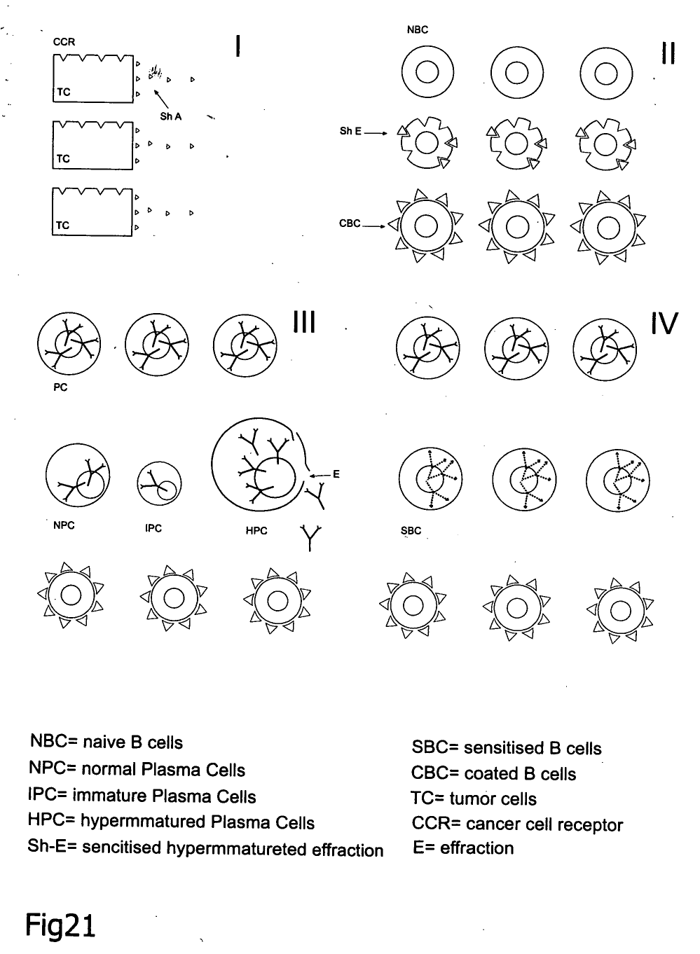

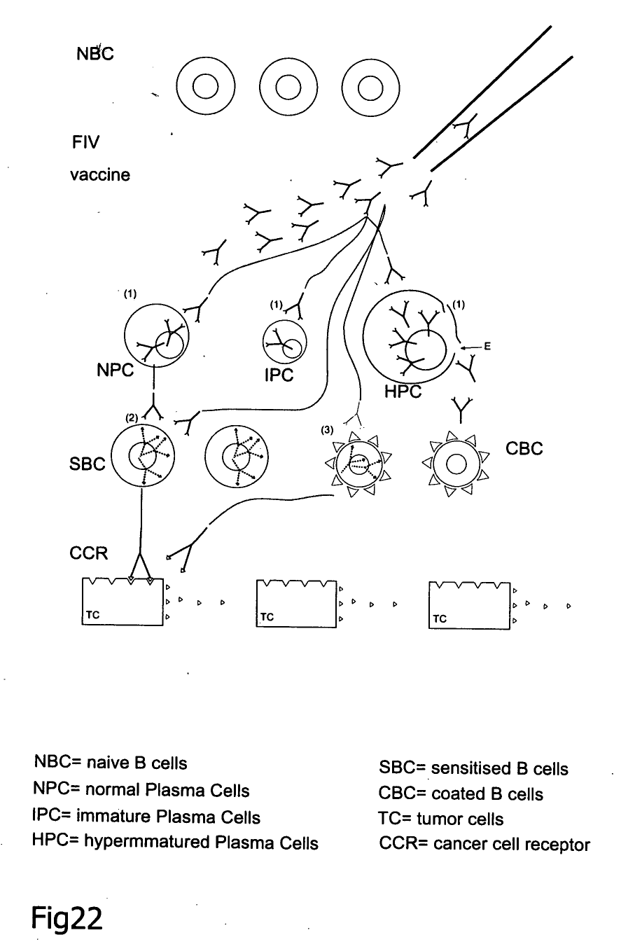

FIG. 3-a1: Compact fields of tumor cells (squamous carcinoma of lung) stained positive in IHC with the use of Fill as Primary Antibody. The unstained pulmonary surrounding tissue very neatly delineated.

FIG. 3-a2: The tumor regions are confined by fields of compressed lung tissue in which are conspicuous Plasma cells of unusual small size. In the most mature of these cells is present intense specific staining. Alveolar empty sacks are still visible.

FIG. 3-a3: Scattered damaged tumor cells including in the field a few positive stained cell with aspect of old Plasma-Cells in course of degradation.

FIG. 3-a4: Mature Plasma Cells in compressed lung alveoles intermingled with smaller cells of the same type in course of decomposition. Positive Immune Histo Chemical Staining of the respective cells in the staining with FIII Primary Antibody of the reaction.

The Primary Antibody of IHCS has been described in previous communications as the novel antibody FIII. FIII mimics the molecular structure of cancer autocrine transformed Growth Factors and may serve as staining agent by binding the respective cellular receptors.

The anatomical items display fields of tumoral tissue diagnosed as Squamous Cell Carcinoma (of lung) and Plasma Cells isolated or in groups located peritumoral. The variation in size and additional morphological changes suggest that the Plasma Cells of this case represent a range of cells of different ages whose development has been arrested or are in a functional blockage.

The IHCS shows the staining of tumor cells with FIII Abs as usual. In addition there is positive staining of Plasma Cells, of the polynuclears marginating the blood vessels and of groups of normoblasts. Whereas the tumor staining is consistent with the putative ligand role attributed to FIII, the staining of the Plasma Cells is a paradox that could not be explained by the binding of receptors of the same type as those expressed on cancer cells.

The unique possible explanation of Plasma Cells staining is the presence of products of secretion whose receptors are in antisense with the exogenous FIII. These cellular products, either antibodies or adhesion Ig-like peptides represent the cellular reaction of a susceptible subset of B lymphocytes to cancer shed antigen.

The staining of Plasma Cells by FIII demonstrates that the new formed Plasmocytes have reacted to cancer antigens by preparation of the basic machinery of secreting antibodies. In the absence of ligands of transmembrane Plasma Cell receptors the release of antibodies is blocked and the immune system enters in the special state of unresponsiveness named PREMUNITION. Although the process of humoral defense is not accomplished to the end, the leakage of some antibodies in the circulating humor may occur and be pinocytosed by polynuclears.

The positive IHCStaining of Plasma Cells of this case suggests that the immune competent cells of a cancer carrier may undergo preimmune changes with or without minimal serum expression. However the susceptibility to antigen of the type FIII is increased and may lead to a huge production of antibodies anti-FIII Abs.

The findings reviewed in the present study point out to the theoretical possibility of a test for cancer detection based on the positive IHCS of Plasma Cells with FIII Abs.

FIG. 4-a: Experimental myeloma in mouse, one month after inoculation. Maximum of development compatible with life.

FIGS. 4-b and 4-c: The same tumor, treated by vaccination with FIV Sheep at one and respectively two weeks after inoculation.

FIGS. 4-d and 4-e: two lots of mice inoculated with Myeloma. The anticancer treatment (vaccination) was applied two days after the clinical appearance of the tumor.

FIGS. 5-a, 5-b, 5-c, 5-d, 5-e, 5-f, 5-g and 5-h: microscopical examination of the anatomical samples of FIG. 4.

FIG. 6-a: Rabbit's thigh, tumor invasion (VX2), six weeks after inoculation Control case. CI on 20 Feb. 1997.

FIG. 6-b1: CT on 20 Feb. 1997 of a quite extended tumor (VX2) on a rabbit right thight. The tumor was inoculated a month ago. Start of treatment by FIV Sheep vaccination for the last week.

FIGS. 6-b2: The same model, three month later, on 29 May 97. Clinical disappearance of the malignancy, confirmed by CT imaging.

FIGS. 6-c1: CT on January 1997 of a rabbit inoculated with VX2 in October 1996, carrying voluminous tumor a few weeks later and vaccinated repeatedly with FIV Sheep. At the first CT, on January, the tumor already shows extended calcification of tumor tissue and improvement in clinical condition of the model.

FIG. 6-c2: the successive CTs performed on March 1997 and on December 1997.

FIGS. 6-c3 and 6-c4: show an increase in the calcification of the tumor localization together with a reduction in volume and a sharper delimitation of the bords of the stone-like calcification. At the pathological examination; complete regression of the tumor.

FIG. 6-d: Treatment of VX2 tumor with an increased dose of IV Vaccine. Energetic reaction of tumor regression and its replacing in most of the areas by fat tissue.

FIGS. 7-a and 7-b: Not treated cases of VX2 tumor (control), 12 weeks after inoculation in thigh muscles. Spontaneous death. Metastatic spread in lungs. Fixation of anatomical items in formalin.

FIG. 7-c: Metastatic spread in lungs and spontaneous death at 10 weeks after inoculation. Lung before formaline fixation.

FIGS. 7-d, 7-e, 7-f, 7-g, 7-i and 7-j: Whitish spots on lungs of treated (vaccinated) rabbits VX2 carriers. Since the vaccination was applied late, it was ineffective in accomplish the regression of primary tumor. However it was effective in destruction and sometimes cicatrisation of pulmonary metastases.

FIG. 7-h: Photograph of non-fixed material of a ‘late-vaccinated’ tumor carrier.

The big primary tumor is necrotic at a great extent but its consistency is saved and no anatomical regression is possible. On the other side the lungs show only traces of the past tumoral metastases as whitish spots.

FIGS. 8-a and 8-b: VX2 in rabbit, intra alveolar metastases in the post vaccination (treatment) period, at two degree of magnification, H/E.

FIGS. 8-c and 8-d: pulmonary metastases, three weeks after the therapeutic vaccination. Intra alveolar tumor cells in apoptosis. TUNEL.

FIGS. 8-e, 8-f and 8-g: apoptosis, at various degree of magnification of intrapulmonary regions invaded by tumor metastasis (TUNEL staining)

FIGS. 9-a, 9-b, 9-c and 9-d: metastatic spread at various magnifications of tumor cells VX2 in the alveolar walls and compact agglomerations. Identification of tumor origin by the generalized apoptosis produced 2 weeks after ‘vaccination’ with FIV (TUNEL staining)

FIGS. 10-a, 10-b, 10-c and 10-d: The metastatic tumoral tissue destroyed under the effect of anti cancer vaccination is replaced by conjunctive (scar) tissue whose presence is demonstrable by Van Gieson staining.

FIGS. 11-a and 11-b: Hepatoma in rats, on living models, respectively, four and two month after inoculation.

FIG. 11-c: Hepatoma in rats, four months after inoculation, exposed and photographed before fixation.

FIGS. 12-a and 12-b: pulmonary metastases of hepatoma, before fixation.

FIGS. 13-a, 13-b and 13-c: Microscopy of Hepatoma, in rat. Control Case, 3 magnifications. H/E.

FIGS. 14-a, 14-b, 14-c and 14-d: Explosive apoptosis of tumor cells, 3 weeks after vaccination with FIV Bov.H/E

FIGS. 15-a, 15-c and 15-d: TUNEL confirmation of generalized apoptosis after vaccination with FIV Bov. FIG. 15-b: Apoptosis in hepatoma after anti tumor vaccination with FIV Bovine stained H/E.

FIG. 16-a: Hepatoma (Control) stained with Mucin-Carmin.

FIG. 16-b: same staining at the surgical scar 4 weeks after surgical extirpation of the tumor.

FIG. 16-c: same staining of the tumor remnants left in the body of tumor carrier.

FIGS. 17-a, 17-b, 17-c and 17-d: Hepatoma in Rat, case 2112. Sacrifice 4 weeks after vaccination with FIV Bov

FIGS. 18-a, 18-b, 18-c and 18-d: TUNEL staining of Lung sections of rat carrying hepatoma, treated with active immunization with FIV Bov.

FIG. 19-a: Abdominal Scar after surgical extirpation of hepatoma tumor one month after vaccination with FIV Bov.

FIG. 19-b: the tumor extirpated, is of bilobated shape and 19 cm in length.

FIG. 20-a: Vaccination of a rat carrier hepatoma with FIV Bov immobilized on Polystyrene beads. Generalized destruction in a few hours of all the cancer tissue occur explosively 13 days after the Primary Vaccination.

FIG. 20-b: Hepatoma in rat at four months not treated. Exposed for control of ths size of the growth of 20-a.

FIGS. 20c and 22d: Macroscopic magnification of 20-a.

FIGS. 20-e: Macroscopic magnification of Control case (20 b)

FIGS. 20-f1, 20-f2, 20-f3, 20-f4, 20-f5, 20-f6, 20-f7, 20-f8, 20-f9 and 20-f10 microscopic details of the tumor destruction in the of combined use of Polystyrene adjuvant.

FIG. 21: Scheme of development of premunition in cancer.

FIG. 22: Scheme of production of FV in cancer carrier by anti cancer vaccination with F IV.

### DETAILED DESCRIPTION OF THE INVENTION

A great part of the medical means proposed as treatments against cancer are efficient when experimentally checked. Unfortunately the accompanying restrictions make their therapeutic use practically irrelevant or of an interest limited to animal models.

The repetition of biological treatments with xeno proteins inevitably induces in the host strong immunological adverse reactions that prohibit the continuation of the cure. The problem of the biological treatment of cancer cannot be solved with a single adequate dose of the foreign agent which may in any case be either an under estimation or an excessive nocuous dose.

The restraint of the pathological cellular proliferation in an evolved metazoan is approached in this Application as an artifactual way of defense against the perturbation of the most favorable balance of development pre-existing in the respective organism. The cellular division in a multi-cellular integrating organism is based on an archaically established function whose appearance precedes the clone differentiation of the metazoan organisms. The restraint of exaggerated or disproportionate cellular proliferation depends thus in part on the immune modulation of the growth and forms the background on which the clone pathology may be expressed. ‘Curing the tumor’ in this case signifies a negative selection by which the action of Growth Factors or Growth ligands on susceptible cells may be prevented.

A strategy of correction able to induce the destruction of cancer cells or at least a generalized arrest of proliferation must include a satisfactory response to the issues that prevent Factors III and IV separately from being operational as medical anti-cancer agents.

An important achievement in this context would be the possibility of avoiding the adverse reactions of proteins injected in the organism of the cancer bearer but saving on the other hand the beneficial effects. The raising of respective factors as self-products of the individual to be treated and not as foreign agents would certainly provide a suitable solution.

The variable domains of FIII and FIV are complementary and of opposite electrical charge. FIV as produced against the variable region of F III is supposed to be provided with functional properties of an opposed symmetry to FIII ⁽⁴⁴⁾. The reverse procedure is theoretically also possible. Using as antigen FIV antibodies, or better still their Fab′ fraction, a susceptible recipient may produce antibodies which have most of the structures of F III, and are respectively very like but not identical to transformed autocrine Growth Factors of cancer cells.

The raising in the organism of a cancer carrier of antibodies of this type changes basically the immunological relation between the cancer growth and its carrier and may provide a satisfactory medical solution for the treatment of the condition.

FV (F5) is a novel antibody raised in, the organism of the cancer bearer by the active immunization of the host with F IV Abs that serves as antigen. The affinity of F5 for the trans-membrane domain of the cancer cell receptors is proved by the positive staining of receptors on sites of other species than the original species used for production of the antibody.

However, despite its isogen nature, and the supposed similarity of the variable domain with the Growth Factors, the antibody F5 is quite different in structural organization and size of the original cancer autocrine ligands. The complete internalization, or at least the integration in the cancer cellular metabolism, as a complete and intrinsic physiological process can not be performed, although the replacement of the genuine Growth Factors of cancer cell receptors with this ‘surrogate of ligand’ represented is made under the pressure of the continual production of this antibody.

The similitude of the procedure to vaccination or treatment with monoclonal idiotypes raised or directed against certain TAAs is only an apparent resemblance. A leading criterion of the modern anti-cancer vaccination consists in the blocking of epitopes of TRK axis or of other cellular paths by monoclonal antibodies and thus preventing the physiological cellular nutrition. This limitation is not relevant in the case of F5. Antibody in which the circulating amount and diversity of idiotypes of F5 is adjusted by a process of immunomodulation in which the most important role is directed in situ by the tumor. Rather than an effect induced by an external monoclonal antibody to a particular TAA this type of active immunization reminds older attempts of anticancer treatment by blockade of multiple receptors of Growth Factors ⁽⁴⁷⁾ but with a different kind of ligand whose production is in this case auto-regulated.

The changes induced by vaccination with FIV in a cancer carrier, start in about 15 days in primary tumors and in less than that in metastasis. The diversity of patterns of reaction includes (i)) apoptotic destruction followed; by total absorption or conjunctive tissue repair and/or (ii) replacement of tumor by non functional (non viable) cells maintained indefinitely in a non proliferative status quo. In both these occurrences the sensitivity of tumor tissue to chemo or radiotherapy even in minimal doses increases remarkably. However is only a theoretical construction to ascribe a therapeutic effect like this to the effect of polyclonal antibodies without a previous factual grounding.

There is no proof of existence of common antigens of the variable domain of FIV Abs and of the receptors of the transformed Growth Factors of the cancer carrier. Without the evidence of such epitopes it does not seem a real possibility that the immunological system of the host may produce a detectable reaction of cross-immunity unless there exists an intermediate mechanism of amplification for the immunological reaction and continuation of the effect on its own.

However, the anti cancer effect by in situ production of F5 is a reproducible evidence if some biological requirements are fulfiled.

One is the cross immunity of GF receptors of the host of with F IV antigens. The immunological background that makes possible an immunomodulatory relation after the vaccination with FIV modulation, requires the development of the state of premunition.

Premunition is defined as a non-specific state of defense supposed to last as long as the pathogen agent is present in the organism of the host ^{(34,71,72,76)}. Known as a mark of some Communicable Diseases, the existence of premunition is however accepted and it is ascribed an important role in some viral feline infections or parasitic diseases.

In modern immunology the concept is controversial and considered obsolete by some modern immunologists. The main criticism emanates from the difficulty of finding serologic or other kinds of evidence for its accreditation, except the imprecise clinical observation.

The demonstration of premunition in cancer presents special aspects. The cancer, as an irreversible and progressive condition cannot display a measurable “immunity with holes” as in syphilis or other infectious conditions. Secondly, the border between premunition and the tumor immunological tolerance is untraceable. The activation of the immune system in cancer differs essentially from the active immunization against other agents, as in Communicable Diseases or aggression by foreign proteins. The cancer immunogens appear in the organism like the paternal antigens in pregnancy, in infinitesimal quantities that increase slowly with the development of the growth, either physiological or by proliferation of cancer cells. Throughout the time that the immune tolerance is maintained and the increase in the titer of the new factor does not modify a certain established equilibrium a reaction of rejection is prevented.

The absence of rejection does not signify, however, that the cancer is exempted of a host immune reaction but only that the immune reaction may not be apparent A metazoan organism eventually discharges the products of the metabolic wasting and secretion in the ‘milieu interne’ and in the general circulation. The cancer cells, whose rate of proliferation determines a high metabolism and rich secretor activity, make no exception of this behavior. The difference is that the cancer products, originating from transformed cells carry non self antigen determinants. By definition, these products are recognizable by the host and supposed to raise a serological reaction unless a special mechanism prevents the expression of this issue at least partially and for a limited time. This is precisely the case of the virtual immunity discussed above under the name of premonition.

Recent immunological evidence ⁽²⁰⁾ completes the findings of the last two decades in respect to the neuro-physiological changes induced by the secretor products of cancer cells on Neuro Muscular Junction (NMJ). According to Patents on this matter registered in the State of Israel, United States and France ^{(12, 14, 15, 16 and 17)} the cancer shed antigens may induce fundamental changes in the activity of NMJ of the frog known as Cancer Electromyographical Evoked Pattern (CEEP). Although the use of the peptide product of cancer secretion (Cancer Humoral Factor, CHF) as antigen may raise a strong humoral reaction in an allogenic host ⁽²¹⁾ the immune status of the tumor carrier apparently is not changed.

The causal agent of the premonition in cancer in the human cases examined is the Cancer Humoral Factor as further findings have established. The confirmation of this immunological relationship was provided by the immunohistochemical staining of human cancer with F III Abs as Primary Antibody. The examination of samples of cancer tissue in a case of Lung Cancer Squamous Carcinoma ⁽²⁰⁾ shows that F III Abs, reagent is staining positively the Malignant Growth and also the hypo and hyper maturated Plasma Cells in the surrounding normal tissue (FIG. 3-a, b, c, d, e, f).

Lung Tumors are particularly known to secrete an unusually high number of peptides provided with cholinergic proprieties. In order to explain the staining of the two targets by means of the same Primary antibody, one has to admit that under the effect of cancer shed antigens a contingent of B cells underwent Plasma Cell transformation and pre-formed as usual the antibody known by the name of F II Abs ^{(21).} However, the Plasma Cell metabolism is arrested in a phase close to the release of the antibody or even earlier than that, probably due to a deficiency of transcriptional repression of BIMP-1 and/or XBP1^{(1,63,66,67)}. According to K Jerne “Idiotypic Network Hypothesis” a generation of antibodies existing in a host, in the case of cancer the FII, packed in Plasma Cells may virtually promote a new anti-sense generation of antibodies. The switch is triggered in this occurrence by vaccination with FIV antibodies that has resembling physical features with F II. The co-stimulatory signal added to Plasma Cells from outside, permeates the membrane and induces an energetic cellular change accompanied by a massive release of sequestered FII Abs. The addition of this endogenous immune stimulation is in the same direction with the production of FV as the result of challenge with FIV.

In this interpretation, the breaking of, the tolerance for tumor is dependant on the accumulation in the competent cells in a latent period of a sufficient amount of epitopes of tumor receptors. At a certain moment or under the influence of a certain extern trigger the immune state of the tumor carrier may switch from premonition in a humoral reaction of antibody production whose variable domain is similar to transformed Growth Factor(s) of the host's tumor, carried by FIII.

It is understandable that the active immunization could not be created in the physical absence of the tumor. The tumor presence as necessary for treating the cancer condition is not a play on words but an irreplaceable factor in the process of defense. The prevention of the condition is not possible without cross immunity between the vaccine and the host, and the dross immunity is only possible with instauration of cancer premonition. In the absence of this state no significant immunological changes may be produced by active immunization with FIV. In a previous experiment, 4 weeks' vaccination with F IV did not prevent the successful transplantation and development of ascitic tumor and hepatoma, while the same vaccination, after the appearance of the tumor, exerted the therapeutical effect expected, as shown further on in this Application.

(Manolescu, Terbea, Moraru, Calmanovici, Unpublished Observation, Bucharest Institute of Oncology)

The examples presented in this Application refer to the use of an immunological agent raised on different animal species for the treatment of cancer regardless of the histological types. The achievement of a similar result with such distinct variables suggests that the principle of the process presents more importance in this case than the diversity of the parameters. The treatment in this case is not limited to particular cases but may serve as a basic system of reference for any tumor, in human or animal models.

The other issue that contributes to the amplification of the reaction is the special relation of immunogen to competent cell that characterizes this type of active immunization

The antibody response of competent B cells is elicited by the challenge of the Immunoglobulin membrane receptor with the template of the immunogen that serves as antigen and which is usually the pathogen factor.

The vaccination with FIV is particular in this respect. The antigen is formed by a population of Ig G antibodies, some of which carry an Ig-like domain supposed to induce the production of the novel F5 antibody.

The vaccine of this immunization, FWV is actually the anti-anti-antibody of FII ⁽²³⁾. As specified in the claims, of the Patent Application for ‘Production of Competitive Antibodies to Growth-Cholinergic Receptors’ the idiotopes of FIV Abs have variable domains with the same functional properties and a similar molecular structure as the Antibodies to Cancer Humoral Factor (CHF) described in the Patent Application for ‘Preparation of Idiotypic Antibodies to Cancer Humoral Factor (CHF) or Factor II ⁽²¹⁾. The molecular structures that challenge the B cells of the host tumor in this manner are a quite far projection of the tumor receptors that served for the production of the vaccine through three generations of antibodies.

Despite the distance that separates the tumor that served initially for the preparation of FIV and the tumor to be presently treated, there are a number of points of encounter that synergize the effect of these two factors.

The specific answer of the immune system may be amplified considerably by a product of secretion of cancer cell which coats the B forming antibodies. An important place is occupied in recent literature by the adhesion factors which in part are produced by the cancer cells themselves as expressing Basigin, a product of a super immunoglobulin family ⁽³¹⁾ known also by other names. Many of the adhesion factors discussed have an incomplete immunoglobulin structure, called Ig-like. The domains of these structures carry complementary sites which may represent an archaic receptor for immunoglobulin^{(13,28)}. As a consequence the antibody production could be very much enhanced by the encounter with an adequate antigen. ⁽⁵³⁾

In this particular case, the external antigen FIV enriched with the antibody content of Plasma Cells or Plasma Cells in formation proceeds as an immunoglobulin with free variable domains able to bind additionally to complementary sites of competent B lymphocyte, a situation which does not exist in the routine active immunizations. As a result, in the case of a cancer carrier, the production of the novel antibody F5 increases exponentially, not dependant on the second immunization and appearance of memory cells⁽⁷⁴⁾.

The novel antibody produced by active immunization with FIV and named generically FV is supposed to have a molecular structure of the variable domain similar to FIII Abs and to fulfill almost identical functions. FV just as FIII is supposed to bind trans-membranous sites on the receptor of GF of cancer cells; however, the two factors are distinguished by an essential attribute. As a foreign protein FIII is not compatible in humans with a treatment of cancer that requires the repetition of the antigenic challenge. In contrast, FV Abs as an endogenous factor does not differ immunologic from the physiological ligands of the species. Since the reaction is auto-maintained as long as the tumor exists in the organism, there is no danger of raising detrimental allergic reactions over the first one or two inoculations with the foreign protein F IV.

FV Abs justifies its appellation, “Surrogate of ligand” both philologically and scientific. The novel antibody is produced as the anti sense of a foreign protein that mimics the cancer cell receptors. FV, as a mirror image of this factor is provided with the necessary structures for binding the trans-membrane receptor sites just like the wild ligands and even to dislocate them under the pressure of a high concentration. However the steric conformation of the factor must prevent the complete internalization and integration in the metabolism of the cancer cell. This non physiologic issue stops the metabolic support of a cell in active proliferation, especially in those with high metabolic needs such as the cancer cell. The consequence of the ‘cell starving’ is the arrest of cellular proliferation and for most of the cells, death in apoptosis

The developments that follow the vaccination with FIV may vary in respect to the size, weight and the age of the tumor. In all the occurrences, the proliferation of tumor cells is stopped.

The image predominantly found is extended apoptosis of cancer cells, characterized by disappearance of the former tumor or its reduction in non-recognizable debris. However, in case of large tumors (e.g. gigantic hepatoma), the tumor, formed by non-viable and ‘dormant’ cells may be maintained in a conjunctive wrapping indefinitely.

The surgical removal of the bulk of the tumor, even leaving a part of it in place and closing the incision leads to destruction of the remaining tumor tissue and healing of the wound in an amazingly rapid speed (2-3 days).

A special consideration is due to the comparison between the treatments of the tumor in human versus animal models. Usually, in humans, a malignant growth that weighs more than 1/80 of the body weight is not compatible with the life of the individual. This is riot the case in animals in which the weight of some tumors can reach impressive dimensions if related to the weight and size of the carrier. In some cases presented further on in this Patent Application the tumor may reach as much as ⅕ of the total weight of the animal. Related to humans, this dimension is an inconceivable monstrosity.

So far vaccination of tumor carriers with FIV has been practiced exclusively on cancer in animal models. The theoretical considerations lead one to expect a greater efficiency in human practice than in animal experiments. This prediction is supported firstly by the slower rate of the growth of the tumor in human than in animal thus affording time for immunological developments. Secondly, the cover of efficient immune therapy offers additional therapeutic possibilities. The surgery of very large tumors in this case may be combined with chemical and radiological treatment in unusually low doses, harmless for the patient, but sufficient to prevent the danger of metastasis. Another group of cancers expected to be especially prone for this type of therapeutic vaccination are the hematogen tumors in which the microenvironment is not expected to support the tumor escape mechanisms.

Theories of biological sciences can only be validated by experimental confirmation. There is no substitute for justification of a healing product other than the healing fact itself and its reproducibility in the same circumstances. The implications of this presentation assume that the non-specific immune reaction induced in cancer patients, is efficient in any histological type and in any extent of the tumor, including metastases. The healing is possible by regression of the tumor or in case of an already prominent tumor by partial surgical elimination or destruction with or without adding chemo or radiotherapy in low dosage

The medical criteria required for control and confirmation of a cancer healing schedule should be no less exigent then those established for the acceptance of cases of spontaneous cancer regression in humans. One advantage in favor of checking treatments in experimental transplantation of cancer is the possibility of surveying the evolution of the tumor starting from point zero. The examples that follow illustrate the effect of the cure with F IV Abs and stress the similarity of action of the agent produced by the same procedure in animals of different species, respectively sheep and cows. Secondly since the curative effect of the agent is not restricted to a certain tumor it may be used for treatment of tumors of different histological types and in various stages of development. And finally, by analogy with the assessment of the spontaneous regression of cancer in human, the types of malignancy to be treated were chosen as those in which objective and measurable criteria such as radiological visualization, serological tests or survival times are available.

Rodents, especially mice were used extensively in experimental oncology. The easy manipulation and objective facilities of experimentation turned the mice into a preferred model for studying the cancer. However, a serious limitation of immunologic studies on mice is that in general the times of tumor development after inoculation and the reaction after the active immunological procedure are too short to enable an observation that may be transpolated to human long lasting tumors. In this respect, it has been noticed that from the variety of tumors experimented on mice, Myeloma is a convenient model for stressing the sequence of times in the effects of vaccination on the tumor development.

The findings of the treatment are evidenced in FIG. 4-a,b,c,d,e and FIG. 5-a,b,c,d,e,f,g and h for the microscopy. Macroscopically, a scale of arrests in the tumor development appears when the non specific FIV vaccine is applied or differently a utter difference is; noticeable when the maximum growth of the tumor is unrestrained by vaccine-treatment.

Since the extent of the arrest is dependent on the interval between the inoculation and the date of vaccination, the experiment is somehow hindered by the superposition of the ‘tumor time’ with ‘survival time’ of mice carrying myeloma. However, the earlier vaccination of lots of mice and evaluation in parallel of the tumor development in control mice enables the obtaining of basic evidence regarding the restraining effect of the active immunization of tumor growth, as shown in FIGS. 4 and 5.

The microscopic examinations of treated and untreated tumors are supported by the conclusions drawn from the macroscopic observations in question. The standard staining by Hematoxilin/Eosin certifies the histological nature of the tumor. TUNEL, as a specific staining for Apoptosis, shows the spread in the area of the cancer tissue in various stages of development. Nonetheless, animals with the tumor arrested in development for many weeks, show a strong tendency to develop fibrotic and conjunctive transformation of the destroyed tissue which stains in red with the specific Van Gieson's reaction.

Another tumor chosen for the experimental requirements of this Application was VX2. This tumor, defined histological as anaplastic carcinoma was isolated in the UK in rabbit during the second World War under the name of V2, but because of the negative connotations with the German rockets which bombed England at that time, it was renamed VX2. The tumor has been preserved since then by transplantation on White New Zealand (WNZ) rabbits and more recently saved by the method of cellular preservation in deep freezing. The tumor may be inoculated and produces growths in WNZ rabbits in any place. The standard site in this Patent Application was the deep layer of the thigh deep muscles. The tumor, inoculated as 1,000,000 cells per dose, becomes clinically evident after approximately 4 weeks and invariably kills the rabbit in another 8-12 weeks in a state of respiratory insufficiency and advanced wasting. The tumor is highly metastatic by lymphatic dissemination especially in lungs (FIG. 7-a, b and c) however this preference is determined by the local circulatory organization and not due to tissue affinity. The arguments for choosing this model for the study of the treatment with F IV vaccination were (i) the large proportions attained by the primary tumor thus facilitating the radiological follow up, (ii) the invariable and rapid association with pulmonary metastases, which offers a pathologically objective evidence of the tumor spread or regression and finally⁽¹⁸⁾ (iii) the relatively long survival times which fit the necessities of immunological observation versus the short time of survival in most of rodents tumors.

The principle of the treatment consisted of Active Immunization with purified Ig G of FIV. The product used in the present case was raised in sheep.

The NZR inoculated with VX2 ⁽²⁵⁾ were divided into small groups of 3-4 animals which differed by the length of the interval between the zero time (the time of tumor inoculation) and the earliest possibility of performing a clinical diagnosis of the tumor by palpation.

In order to estimate the effects of the immunological treatment alone without inference of other therapeutic factors, the addition of chemotherapy, radiotherapy or surgical procedures was avoided. All the cases were treated with an immunological agent from the same batch, in this case purified FIV Abs raised in sheep.

The treatment itself, was carried out by intra-dermal vaccination of the of tumor-bearer rabbit with 15-20 mg antigen. F IV Abs solved in 1 ml saline solution and emulsioned with an equal volume of Complete Freund Adjuvant. The primary vaccination was performed at slightly different intervals of time with regard to the data of tumor inoculation, in order to equalize in general the differences in tumor growth and infiltration into the surrounding tissues. The surviving cases of the group received in 3-4 weeks, a second vaccination with the same immunogen. The procedure was repeated over the following months if the animal survived and its general condition allowed. The animals were weighed regularly and the tumor growth was checked up clinically and by Computerized Tomography examinations.

Until recently the radiological assessment was not a part of the routine methods of examination of experimental tumors in laboratory animals, either because of the technical limits of classic radiology in the visualization of soft tissues, or because of the small dimensions of the tumors in rodents. Nevertheless, the appearance of new radiological technology such as CT and MRI supplies an answer to both these impediments and makes attainable the difficult task of evaluating in vivo the volume and structural changes of tumor tissue submitted to the effect of a certain therapeutic agent The increased imaging possibilities of Computerized Tomography together with the facilities for the examination of bigger animals such as the rabbit were particularly advantageous in this Application, objectifying the changes in time of the tumor under the effect of vaccination with FIV Abs. The importance of the imaging examination in this study is evident. The same as in the criteria used to assess the ‘Spontaneous regression of cancer”, only the serial radiological image may confirm the succession of the stages of the condition and negate possible errors in identification of the tumor carrier^{(26,32,33,40,57,73,77)}

The rabbits, inoculated with VX2 underwent serial CT examinations at MAR INSTITUTE in the BNAI ZION MEDICAL CENTER. The result of the examinations, interpreted by E. Barmeir and S. Croitoru are detailed in Appendix IV of this Patent Application.

The animals examined were maintained in cages, slightly anesthetized or not, according to the circumstances.

The clinical features of rabbits inoculated with VX2 and treated with FIV Sheep did not develop strictly uniformly and individual differences existed despite equal conditions of experimentation. However, considering that in 100% of cases the condition is inevitably lethal, the statistics should be interpreted in the light of the biological trends they express and not as a simple numeric relationship.

The CT examination of tumor growth in untreated rabbits showed extension of the tumor on almost all the width of the thigh region. The tumor is not homogenous and displays larges zones of lower density bordered with extended margins of calcium deposition (FIG. 6-A).

The rabbits inoculated by VX2 and treated after the appearance of the tumor with FIV Sheep (purified whole antibodies) developed the tumor condition in clinical patterns recognizable and measurable by physical parameters

A first group, which represents about a third of the number of animals of the lot, healed clinically, put on weight and survived exempted of pathological findings a year or more after the tumor had reached its maximal size.

The CT, the sacrifice of the animal and the pathological examination revealed that the lot consists of two sub-groups exhibiting different situations. In one, the tumor disappeared in approximately 6 weeks leaving in its place a teased accumulation of fat. At the pathological examination the place of the malignancy was marked only by a few conjunctive elements and cellular debris. (FIG. 6- B1 and B2). In the second case, the place of the former tumor was occupied by an intensely calcified sphere, whose formation was already signaled in the incipient stages. The successive examinations which proceeded for more than a year showed an increase in the intensity of the calcification but a lessening of the diameter and a better delimitation of the border (FIG. 6-C1, C2, C3 and C4). The general condition and weight of the model were physiologic. The animal was sacrificed one and half years after the tumor inoculation. The pathological examination revealed only a fat atmosphere around the former tumor and fibrous tissue intermingled with calcium depositions in the radiological dense region.

The last case demonstrated radiological is shown in FIG. 4, D. The tumor had a quite expansive growth after inoculation but also reacted promptly to vaccination, repeated after a few weeks. Although some tumor rests are still seen in the region of inoculation, most of the tumoral tissue was replaced by fat transformation which occupies a quite extensive volume of the thigh No lung metastasis was found at autopsy, 4 months after inoculation.

The tumor in question, VX2 is early metastasing. As shown macroscopic in all the pictures of FIG. 7 and also by microscopic examination the lungs are a preferential site. The death of the animals is due either to the occupancy of the alveolar surface as seen in FIG. 8-a, b, c and d or to a general wasting and emaciation of the models. However the metastasis is extremely sensitive to the action of vaccination. As shown macroscopic in a series of lung examinations (FIG. 7-d, e, f, and g), the metastases regress almost completely under the effect of vaccination although in most of the cases the large dimensions reached by the primary tumor are already outside the control of immunological active treatment and the life of animal could not be saved (FIG. 7-h, I, j). The destruction of the alveolar lung metastases (FIG. 8, a and b) after vaccination with FIV is produced generally by apoptosis of the tumor tissue as seen in the photographs c and d of the same FIG. 8. The apoptosis of lung metastases has the same or similar aspect with the changes induced in the primary tumor at 2 weeks after vaccination (FIG. 8-e, f and g)

The apoptotic and progressive destruction of tumor tissue of primary VX2 under the effect of vaccination is evident in and FIG. 9-a,b,c d where the process may be followed at different sites and magnifications. Interesting that the apoptotic destroyed tissue is not always replaced by fat but a repair conjunctive reaction may take place as seen in FIG. 10-a, b, c and d.

Hepatoma in rat, is a tumor of spontaneous appearance, isolated thirty years ago in large breeds of Wistar rat. Morphologically, the tumor appears as an unlimited growth of acins of epithelial transformed cells, bordered by a dense conjunctive fibrous texture. The incubation period is usually long, at least a few weeks after inoculation and there are varieties in which the inoculation period is much longer, sometimes as far as 8-10 months. Despite these differences, three characters mark Hepatoma in rat as especially fit for immunological, pathological and therapeutic studies ⁽¹⁹⁾:

(i) the tumor may reach to a enormous size without endangering the life of the animal, (ii) the tumor carrier may survive in good general condition for long terms and finally (iii) the staining affinity of the tumor tissue for mucin-carmin in the primary tumor as well as in metastasis makes possible the identification of tumor debris after the destructive effects of the immunological treatment.

Most of the transplantations which served as illustration of this Application were made on lots of Wistar rats but successful transplantations on other rat strains are possible, pointing out that the tumor is not strictly species specific. The tumor may be reach large, even huge dimensions (FIG. 11-a, and b) and pulmonary metastases are a rule (FIG. 12-a and b). The microscopic examination is displayed in three degree of magnification (FIG. 13-a, b and c).

The therapeutic vaccination of hepatoma-bearing rats was performed with Sheep F IV Abs as in the precedent case of rabbits carrying VX2, but also with FIV of bovine (cows) origin with similar results.

The therapeutic scheme was relatively as simple as in the treatment of VX2 tumor in rabbits. It consisted of an intradermal injection of an emulsionate of 30 mg FIV Bov solved in 1 ml Saline water and mixed with a correspondent volume of Freund Complete Adjuvant. In accordance with the clinical evolution in the following weeks the vaccination was repeated or not. Usually, additional injections of FIV were not necessary. The growth of the tumor stopped in approximately 2-3 weeks after the primary vaccination and was followed either by softening of the tumor bulge and retreat, or partial retreat of the tumor mass. Large, prominent, or even huge tumors may be maintained time at a steady size, without affecting visibly the general condition of the animal.

The microscopic examination by routine staining in small and medium tumors of the tumor tissue, after biopsy or by sacrifice of the animal showed wasted cellular areas mingled with extended regions of non viable or destroyed tumor cells but also abundant conjunctive tissue separating and wrapping the tumor acins (FIG. 14 a, b, c and d). The TUNEL staining two or more weeks after vaccination with FIV shows generalized apoptosis in 100% of the cells of the microscopic field in extended tumor areas (FIG. 15 a, b, c and d) However in some large or huge tumors the microscopic examination also shows cancer cells in arrest of metabolic processes, non viable or dormant. The picture suggests an equilibrium between a population of malignant cells whose potential of growth was suppressed for an indefinite time and on the other hand, the presence of humor factors, in sufficient amount to restrain the pathological multiplication in the whole tumor but not competent to turn this balance into the immunological destruction or rejection of cancer. In practice, this virtual development proved to be the adequate explanation of the state induced by the vaccination with FIV in the case of solid tumors whose size surpassed the capability of the organism to eliminate it. The therapeutic consequence is the recommendation for surgical extirpation of a tumor or more exactly of a large part of the tumor, but not necessarily radical, exhaustive surgery.

The rests of the tumor left in, place or caught in the wound by the surgical suture degenerate spontaneously after extirpation of the bulky part of the tumor tissue. Nonetheless after 1-2 months the histological identification is no longer possible the tinctorial affinity of the cancer tissue left is preserved for long time. In this particular case the histological staining with mucin-carmin enables the recognition and localization of tumor tissue, otherwise completely devitalized (FIG. 16-a,b and c).

Surgery was the oldest of the methods used over centuries for palliative or radical treatment of malignant growths. Although a comment about the insufficiencies of the surgical method in the treatment of cancer is beyond the goals of this Specification it should be mentioned that in the absence of additional cytostatic or radiological treatments the cancer surgery is often followed by local recidivism and metastasis. On the other hand the standard therapeutic doses of chemo and/or radiotherapy generally induce undesirable effects. This generally recognized fact may be modified under vaccination with FIV as stipulated in the following:

the vaccination with FIV alone or combined with surgery of the tumor may lead to destruction of the primary tumor or of residual primary tumor after surgery; it cures the existing metastases and prevents appearance of new metastases after surgical manipulations.

the vaccination with FIV decreases considerably the extent of the danger of metastasis after surgical manipulation. This means that the sudden reduction of the tumor mass in the particular conditions of previous active immunization switches the equilibrium between tumor tolerance and anti-tumor humor factors in favor of the latter as may happen sometimes in the exceptional cases of tumor spontaneous regression. A typical effect of the tumoral destruction by apoptotic transformation is shown in FIG. 17-a,b,c and d.

This issue does not include the hematogen cancer in which the surgical treatment is not possible. On the other hand the contact of cancer cells with the immune agent takes place in malignant blood conditions directly in blood, unmediated. The avoidance of an intermediary microenvironment compartment may prevent an unequal repartition of F5 Abs and equalize with beneficial results the seric concentration of the putative F5 agent.

The pathological aspect that may result following the vaccination with FIV may vary in respect to the size, weight and time of development of the tumor. In all the occurrences, the proliferation of tumor cells is stopped.

The image found predominantly is apoptosis of cancer cells in an advanced stage characterized by disappearance or non-recognizable debris of the former neoplasia. In case of large tumors however (e.g. hepatoma), the tumor, formed by non-viable and ‘dormant’ cells may be maintained in a conjunctive wrapping for long indefinite terms.

The removal of the tumor by surgery, even if leaving a part of it in place and closure of the incision leads to destruction of the remaining tumor tissue. The healing of the incision wound is rapid, sometimes only 2-3 days.

The vaccination of tumor carriers with FIV was only practiced so far in experimental cancer in animals. The theoretical considerations are in favor of a better efficiency in human practice than in animal models. Also, better results are expected in hematogenic (leukemia) than in solid tumors which may use escape mechanisms under the protection of the microenvironment. These predictions are supported firstly by the slower rate of growth in human than in animal thus affording time for immunological developments. Secondly, additional possibilities are offered by the early discovery of the tumor in human. Under the cover of this immune therapy the surgery and the chemical and radiological treatment in low doses are harmless for the patient however the danger of metastasis is prevented.

The method of immunization described in this Application is subject to improvement by refining and better use of technical parameters. An important part depends on the properties of the adjuvant used for potentiation of FIV antigen.

The vaccinations discussed in this Application were carried out using Freund Complete Adjuvant. As judged by the results of the treatment the production of F5 may reach the minimum amount indispensable for inducing cancer regression in histological varieties in which the spontaneous lethality is considered to be 100%. However, whether the routine technique of emulsion and embedding the antigen in mineral oil or saponine preserves accurately the immune value of molecular structures for the purpose of this immunization is uncertain. The particularity to be taken into consideration in this. Application is that the variable domain of the antibody that serves as antigen is expressed on the cancer cellular structures as a receptor for GF. It is of strategic importance for the vaccination whether the immunogenic potential of the antigen is used at a maximum with the conventional procedure, respectively if the affinity of complementary structures exhausts or approaches the limit of possible combinations between two large groups of polyclonal factors in the attempt to find the fittest antigen-antibody coupling.

The use of polymers proved to be an efficient method of increasing the sensitivity of immunological reactions in certain tests of detection.

Polystyrene is a convenient choice for creating solid substrates for the immobilization of protein molecules of antibody type. The adhesion of the Fc segment of antibodies to a Polystyrene ground led to a remarkable increase of the sensitivity of the reactions in immunological procedures of detection; either the binding was either made on ultra flat surfaces, ^{(11,27,29,59)} micronic or submicronic beads with or without previous chemical treatments ^{(68, 69)}.

The structural changes that follow the immobilization of the Fc domain may discover new antigenic sites and multiply by tens and hundred of times the immunogenic reference base.

However, in spite of the successes recorded by the use of polymers in the methods of detection no attempts are known to have been made for the use of polymer adjuvants in the active immunization procedures.

The lapse is not due to an intellectual omission but results probably from the specificity of the process of antibody preparation. The requirements in the case of FV are that the antigen should be an Immunoglobulin and, not less important, to ‘show’ a maximal number of antigenic sites possible. The procedure of changing the molecular arrangements without modifying the molecular structures proved to provide, in this case, the desired result.

The production of antibodies by association of two adjuvants is not a procedure that is used often or at all in immunology. The adjustment of the two agents in the experimental conditions of this Application is carried out by increasing, in a first time, the number of antigenic sites which may have affinity for receptive immunoglobulin structures of B lymphocytes. The preparation of the antigen is completed by the routine emulsion with FCA or saponines and supply of the homogenate to Antigen Presenting Cells of the host. The result of this mixed-adjuvant procedure is the production of the cancer blocker F5 Abs in a titer not reached so far in the active immunization against cancer.

The principle of the treatment described in this Application is the treatment of cancer regardless of its histological nature by occupancy of receptors of GF by F5 Abs produced in situ. The possibility of replacement of the wild ligands by the artifactual F5 product depends not only on the affinity and avidity of the novel antibodies but to a great extent on the ratio of wild and novel ligands, which may influence the speed of the replacement according to the Michaelis-Mentis formulas.

It ensues that treatment of cancer by F5 Abs produced in situ may be carried out by two techniques of immunization. The routine vaccination using adjuvants as FCA in animals or saponines for human patients is satisfactory and may be combined if there is a medical indication with mild conventional chemo-radiotherapy. An additional possibility is provided by the combined use of the two adjuvants described, in an order that favors the maximal use of properties of each of them. The immunological result is an vigorous production of ligands that already reach a high concentration with the primary vaccination without the necessity of the booster. This aggressive humoral effect is however possible exclusively in tumor-carriers due to immunoglobulin specificity of the antigen and can not be raised by vaccination with the mixt preparation, in the non-cancer cases.

The performance of anti-cancer therapeutic vaccination with FIV of this invention in this variant must still be of limited use in medical practice because the intensity of the reaction is not yet sufficiently predictable and controllable. Usually, the fulminant apoptosis of the whole tumor is accompanied by necrosis of spleen and lymphatic ganglions. The rapid absorption of the protein products of decomposition produced in tumor-carrier rat in just 12-13 days after anti-cancer vaccination is leading to the insufficiency of organs of deputation, liver and kidney. The death of the tumor carrier is unavoidable

The dramatic development of the post-vaccine reaction has no precedent in the history of cancer biology. Although the unpredictable death of the subject prohibits presently its use as a medical means its benefits may be inestimable. The present pitfalls regarding the control of the intensity of the post-vaccination reaction are due to the difficulty to make a correct evaluation of the number of antigenic sites, after immobilization of the Fc segment and the difficulty to evaluate at all, the state of premunition that makes this reaction possible without a second immunization. The acknowledgement of the existence of the above two points may promote a solution for an easier manipulation of this type of adjuvant.

The following presentation of Rat Hepatoma treatment by mixed adjuvant vaccination is illustrative in this respect.

The vaccination was carried out in the third month of development of the tumor (rat hepatoma) with FIV adsorbed on a polymer substrate, respectively Polystyrene. According to the protocol, 5 mll FIV in a dilution Ig G 20 mg/mll was incubated with 300 mg. microbeads of Polystyrene at 38 C at 7, 4 Ph. for 48 hours. At the end of this period, the suspension was mixed with an equal volume of FCA. The product was injected intra or infra-dermal, dispersed on as large an area as possible.

No clinical reaction was recorded in the first 12-13 days. At the end of this term, correspondent to the time of releasing the novel antibodies, the tumor started to soften, lost its consistency and became fluent. The animal showed quite rapidly the clinical evolution of a massive toxic state. The development was inevitably fatal in a few hours due to massive autointoxication with the products of decomposition (although the avoidance of death should perhaps be possible by surgical removal of the tumor tissue in due time). FIG. 20 in its subsections shows the anti-tumor effect exerted by the variant vaccination but this effect is also accompanied by detrimental developments suffered by vital organs, blocked by cellular products of decomposition.

The cancer treatment using the agent F5 described in this Application has been shown to be a solution for cancer cure in conditions of varied histological nature and on different animal species. It may produce healing by cancer regression in cases in, which the size or location of the tumor do not present an anatomical incompatibility and makes easier the burden of surgery, chemotherapy or radiotherapy if these procedures are necessary. However, in spite of the evidence of regression of a condition considered otherwise unavoidably lethal, the Application refers to particular cases and can not claim implicitly the same results in cases unverified experimentally. Epistemologically speaking the method may be considered universally valuable only if de facto efficacy of F5 in the treatment of cancer is confirmed by codification of a standard serological test.

An appropriate test for establishing the common denominator of cases which serve as evidence of the present Application and any other cancer condition seems to be the Western Blot, as an examination which may give an objective appreciation of the effects of cross immunization induced by the FIV agent.

The Western Blot has been carried out on animals of the lots used in the experimentation of this Application, respectively rabbits carrying VX2 tumor and rats (Wistar) on which rat hepatoma was transplanted, pregnant mice and rats whose pregnancy was interrupted by FWV vaccination and control cases. The pioneer examination of the sera of cancer patients with this test was performed at the “Institute Pasteur”, Bucharest, by Mihai Danesh, Daniella Botosh and Ana Cismilianu.

The Western Blot actually tests the humoral reactivity of cancer carrier subjects but also other conditions challenged with FIV.

Principally, the Application implies a humoral reaction as a response to vaccination with FIV. This may vary between ‘no reaction’ in absence of tumor to a ‘positive reaction’ in various degrees of intensity depending on the tumor Growth Factor Receptor expression. The objective assessment of the reaction may be achieved by a standard Western Blot. Considering that the epitopes involved in the cross-immunity of FIV belong to molecular structures of the variable domain of the antibody, it follows that a positive answer may be obtained by the electrophoresis of the antigen either of the whole molecule or only the Fab′ fragment and interaction with the serum to be checked. Both options were checked in sera of tumors carriers of this Application treated by FIV Sheep in the case of VX2 and FIV BOV of bovine origin. In addition, an examination was made of serum of control subjects of the same species and serum of untreated tumor carriers

A last category was control sera of hyper immunized normal subjects or irrelevant cases.

The cancer bearer responds to the challenge with FIV with production of reactive proteins detectable by species specific conjugates of tumor recipients.

The immunological response of the cancer bearer is richer and stronger when, instead of the whole molecule, only the Fab′ fragment is used for electrophoresis. The reaction is negative in normal control subjects. A slight, weak, reaction may be present in cases of advanced but untreated tumor or tumors in recipients in which an early immunogenic reaction is raised.

The positive results of the Western Blot in the case of models of cancer, treated in this Application by F IV active immunization, provide an immunological understanding of the clinical and pathological changes described and establish the place in cancer biology of the curative effect of this type of vaccination. Further, the test may be indicative for the immunological status of the patient throughout the treatment. Longitudinal studies in the follow up period after clinical disappearance of the tumor may provide valuable information concerning the necessity for additional radiological or chemotherapeutical treatments.

The presence of a positive Western Blot of the sera of tumor carriers untreated by active immunization with FV points to a natural reaction against the epitopes of Growth Factor Receptors suggesting the installation of a premunition state.

### EXAMPLES

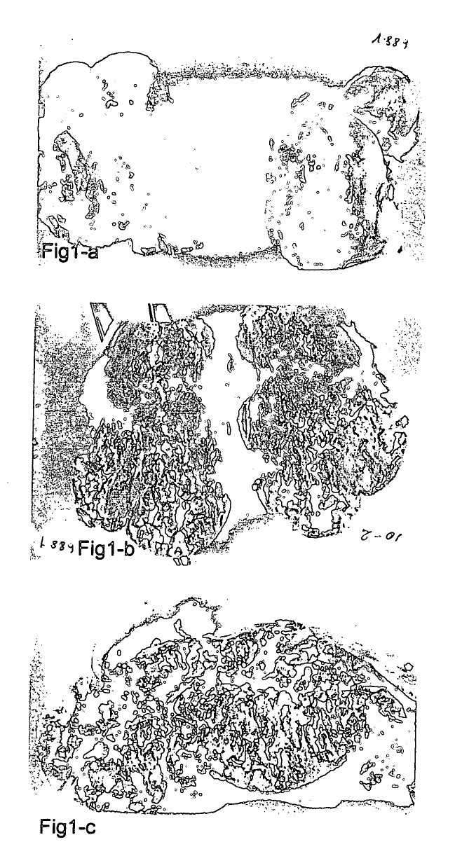

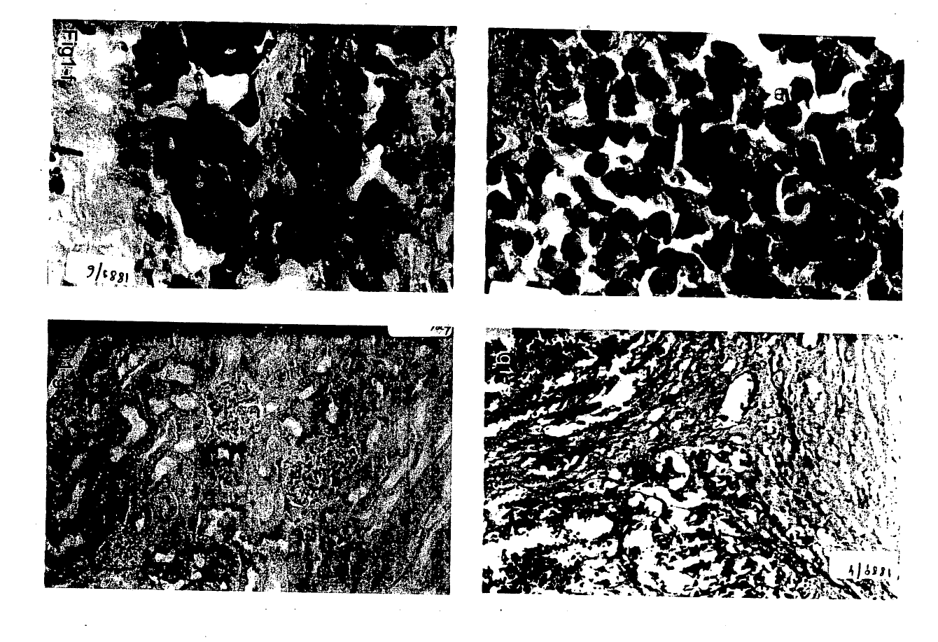

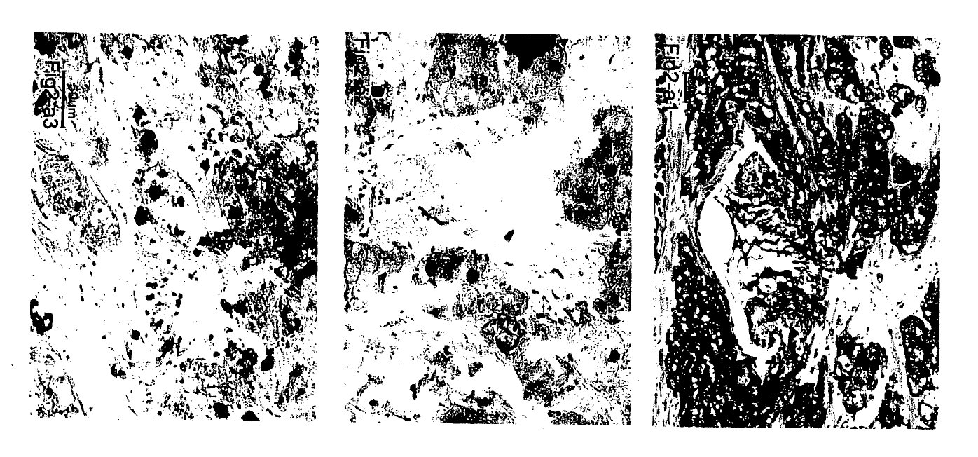

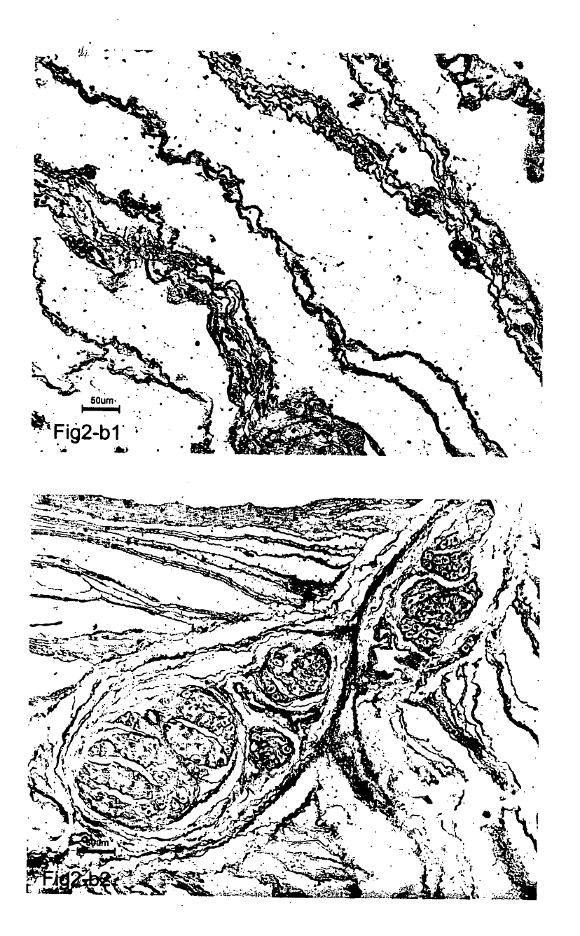

FIG. 1-a, b and c; The treatment of VX2 tumor in rabbit by systemic injection of FIII determineds the binding of the agent on the receptors for autocrine factors of cancer cells and the inducing of apoptotic destruction. On the other hand it continues a productive accelerated growth in lymphatic ganglions (a) and the Primary Tumor (b and c)

The unrestrained growth of tumoral formations and the conspicuous pathological transformations are shown on the above samples. The strong tumoricid action of the FIII agent is thus confirmed but also its improperness for the use in a comprehensive cure of cancer.

FIG. 1-d, e and f: the microscopic aspect (H/E) shows the generalized cellular destruction and cellular rests of the magmatic materiel macroscopically noticed.

FIG. 1-g; TUNEL reaction of kidney. Elimination by urinary tubules of apoptotic rests of the cellular mass.

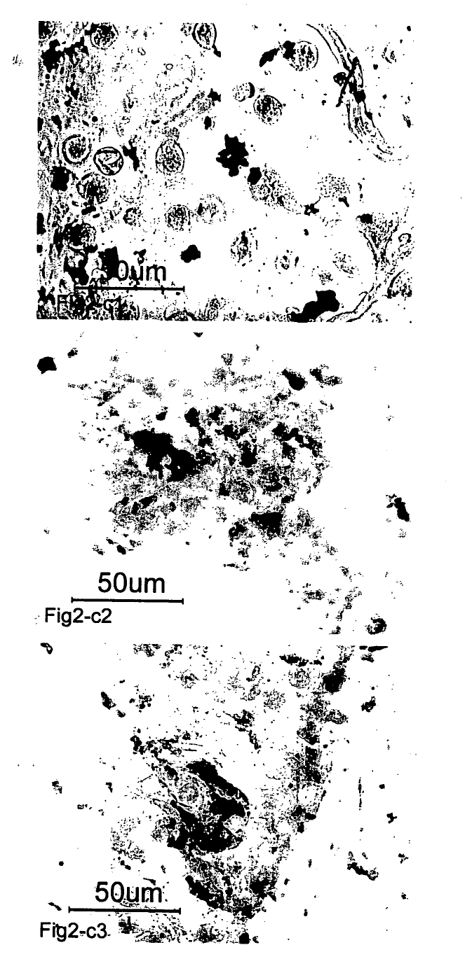

FIG. 2-a1,2 and 3: In the conception of this invention F5 Abs represent an immunological reconstitution of a late member of the family of shed antigens included in the Cancer Humoral Factor of cancer patient's serum. According this postulate, the effects of F5 Abs are supposed to be related to both cholinergic and proliferation function, just as the autocrine transformed Growth Factor of the original antigen.

The cholinergic affinity of F5 Abs is checked in FIG. 2-a by the method of Vital Immuno Staining (VIS) described largely in Application for Patent 164681/84, Israel ⁽²²⁾. The product is checked for its function by its injection into a living model supposed to have appropriate and reactive receptors. One, of the main properties of the neuropeptides is to function as modulators of the neurotransmission and be neurotransmitters by themselves, especially in invertebrates.

In the case of this experiment F5 Abs was injected in the muscles of gastropods (slugs). The models have been submitted to Intermittent Electric Stimulation for 15-30 minutes, sacrificed by formolization at variables period of times (hours) after injection of F5 and processed for histology. The immunostaining procedure was carried out by skipping the time of incubation with the Primary Antibody. Since F5 of this experiment was raised on cow, the Second Antibody used was monoclonal rabbit anti-bovine peroxidase conjugate (Sigma).

FIG. 2-a(1) shows the cephalic nerve ganglion of slug, stained with the standard coloration Hematoxilin/Eosin. FIGS. 2-a(2) and 2-a(3) shows the same region after performance of the mentioned Vital Immunohistochemical Staining. The big size of snail's neuron makes easy the visualization of stained F5 exclusively peri-and intra neuronal. The image suggests that the agent injected in the muscles of the caudal part of the slug's foot found its way to transmembrane neuro receptors of spontaneous active or activated (electrically) neurons. On both photographs are conspicous scattered sites of intraneuronal internalization.

FIG. 2-b 1 and 2: The relation of F5 to the cholinergic effector may also be checked by the direct affinity of the agent to the AChReceptor. A convenient model is provided by the Electric Organ of Fish (in this case Torpedo Electricus) in which a rich presynaptic network transports continuously to the terminal organs the flow of cellular products, including ACh receptors. The positive Immune Histochemical Staining of presynaptic network with F5 as Primary Antibody confirms thus the cholinergic propensity of this antibody.

FIGS. 2-c 1, 2 and 3: F5, as a biological projection of cancer autocrine Growth Factors should be related with receptors on cancer cells that are active in a certain phase of the mitotic cycle. In the absence of a cinematographic technique it would be quite difficult to bring together in a single image a process that occurs in a succession of times. However, this assumption is confirmed in this case by the procedure of immunostaining in vivo. The injection into cancer carrier of Primary Antibody in excess (F5 in rat Hepatoma carrier) enables the visualization of the relevant agent in the cells that enter successively the asynchronous mitosis with the condition that the agent is still present in the organism. Also, as seen in the FIGS. 2 and 3 of this paragraph, as an effect of interference of the agent with the cellular cycle the morphology is profoundly altered and most of the stained cells are already decomposed.

FIG. 3.- a, a1, a2, a3, a4 and 3b, 3c, 3d and 3e show the poster “PLASMA CELLS IN SQUAMOUS LUNG CARCINOMA; RELATION WITH PREMUNITION.”

The positive staining of the tumoral tissue with FIII Abs (Sheep in the case) is consistent with the main thesis of this Application, namely the cancer cells express receptors for shed antigens or antibodies that mimic some of their functions.

The illustrations of the Poster show indeed in this case of Squamous Carcinoma the positive staining of the primary lung tumor. Surprisingly, beside the tumor specific staining expected the pictures showed in the surrounding normal tissues a dispersion of Plasma Cells of slightly modified morphology and staining affinity for the same reagent as the receptor of tumor cells.

The presence of Plasma Cells in Squamous Carcinoma was already noticed in the literature. However the morphological and staining characteristics of Plasma Cells of this case are paradoxal and may be explained only by an immune state of incomplete expression known as premunition. According this Application that is the condition that enables in cancer in exceptional cases the immunological switch of spontaneous regression or the cure of the tumor by treatment with F5.

FIG. 3-f, g: Stained Plasma Cells, in the tissues surrounding the primary tumor, some of them altered or with morphological distorsions.

FIG. 4-a: Control case, no treatment. The tumor reached a maximal development in 5 weeks Spontaneous death in 2 days. Spontaneous necrosis of the distal half of the tumor.

FIG. 4-b: In two weeks after the clinical appearance the tumor has reached approximately the half of its maximum size. Vaccination with FIV slows and arrests at a certain extent the development however the death of the model occurs approximately the same time as the Control case. (Control specimen in the same photo).

FIG. 4-c: Same treatment, applied one week after the clinical appearance of the tumor in paw. The arrest of the development of tumor and necrosis of its distal part start at a earlier stage, however the death of the animal is produced about the same time as the other groups.

FIG. 4-d and e: 2 identic groups of 5 mice, inoculated with myeloma in paw, in respectively right and left hindlimb. The Active Immunization with FIV Abs was performed in the right day of detection of appearance of tumor on two of the mice of each group.

Examination of the groups two weeks later shows a slight difference in color and thickening of the hindlimbs of vaccinated mice versus symmetric limbs non inoculated. The major change is the utter difference between the arrest of tumor development in the vaccinated models and the unrestrained growth of the tumor in the inoculated and not treated animals of the lots.

FIG. 5-a and b: H/E staining of sections on two weeks after tumor appearance and Anti cancer vaccination: undistiguishable tissular structure, probably necrosis coagulation. No signs of viable tumor growth. FIG. 5-c: Van Gieson staining; positive staining at the former site of the living tumor, probably in course of fibrotic organization. FIG. 5-d: necrotic tissue at high magnification, probably former tumor cells.

FIG. 5-e and f: Myeloma at the beginning of tumor growth, HIE.

FIG. 5-g and h; TUNEL staining 2-weeks after vaccination, small and respectively high magnification.

FIG. 6 deals in its subsections with the radiological evidence of cancer, healing/regression as an effect of F5 Abs produced in the organism of cancer carrier.

The value of radiological evidence is the establishing of uncontestable criteria of regression of the condition. The difficulties met in this field have been discussed at large in the Description of the invention. The modern procedures in the visualization of soft tissues are used in the experimental oncology of small volumes and allow a real evaluation of the sequence of times of development of tumors.

These opportunities have been used in a combined study of vaccination under the control of CT providing thus peremptory evidence about the anti cancer therapeutic method used.

FIG. 6 A: Reference. CT image of soft tissues in rabbit invaded by VX2 tumor. Absence of any treatment. 6 weeks after inoculation of 1,000,000 tumor, cells in the deep muscles of the thigh the tumor spread in all the width of the segment forming areas of differing densities and amorphous calcifications, clearly defined. The death was produced in decay and respiratory insufficiency in two weeks.

FIG. 6B1: (case 1897) VX2 inoculation in right thight on Dec. 18, 1996.

CT February 1997: infiltrative process; 2×4 cm, heterogeneous, with internal calcifications and irregular borders.

Vaccinations aZ 20 mg+Freund Complete Adjuvant on February 14, 1997.

Re vaccination Mar. 5, 1997.

Re vaccination Apr. 6, 1997

Clinical disappearance of tumor callousness.

FIG. 6B2: Regression, almost complete of the heterogenic process. Some tissue residue (2×0.5 cm)

No signs of calcification. The rabbit grew in weight from 2.400 gr to 3.100 gr, lived for another whole year and was finally sacrificed.

FIG. 6C1; Rabbit inoculated on October 1996 with VX2. Vaccinated with aZ 20 mg at the end of month November and repetition of the vaccination after three month.

First CT performed on 16 Iannuary 97, second on March 97 (6-C2) and last Decembrie 24, 1997

(6-C3). The tumor, of globular aspect, well delimitated and separated of the normal tissue by a layer of low density, probably fat showed already at the first CT a tendency to circular calcification that, on March; at the second CT, looked as a generalized tumor calcification. At the term of the third CT, on December the rabbit was in excellent condition, grew in weight and showed at the CT examination a slight decrease in the diameter of the tumor, an increase of the density of the calcified tumor and an extended zone of peritumoral fat. At autopsy, in the former place of the tumor were found rests of brown fat, grains of calcium and fibrosis but no tumor tissue.

FIG. 6-D is the last case from the series of tumor carriers that received immune treatment under the control of Computerized Tomography. The tumor was quite well developed 6 weeks after inoculation. The tumor carrier was treated with a double dose of anti Z than usual (40 mg) homogenized with FCA and reactivated after 4 weeks. The immunological response was quite brisk and energetic. CT examination shows replacement of the tumor with a fat, and extended mass in the interior of which are still included a few tumoral rests.

FIG. 7-a and b Inoculation of VX2 in thigh, in rabbit. No treatment. Spontaneous death after 12 weeks. Rich metastatic spread in lungs, well visualized after fixation in formalin 4%.

FIG. 7-c. Inoculation of VX2 in thigh in rabbit. No treatment applied. Spontaneous death after 10 weeks. Photograph of fresh preparation, before fixation. Smaller metastases than in precedent case.

FIG. 7-d, e, f, g, i and j; whitish spots on lungs of treated (vaccinated) rabbits VX2 carrier with some cases as F7-d where are few metastasis, larger than usual are conspicuous. Since the Active Immunization was applied late it was ineffective to accomplish the regression of primary tumor and also of big metastases that continued to, grow. However it was effective in destruction and sometimes cicatrisation of pulmonary metastases.

FIG. 7-h (rabbit 4437) Spontaneous death of a “late-vaccinated” tumor carrier. Fresh materiel of a recent autopsy, before fixation.

The surface of section of the primary tumor presents extended areas of necrosis however its consistency is maintained. Although the regression of tumor is not any longer possible at this size, the lungs show whitish areas relevant for metastases that passed a process of dissolution.

FIG. 8-a and b; intra alveolar metastases, in the post-vaccine period, at two degree of magnification. H/E staining.

The metastases show greatest sensitivity than the primary tumor to the Active Immunization. The effect is at a great extent function of the size of the tumoral mass which may enable or not an easier physical approach of the therapeutic factor to the malign tissue. In accordance with this postulate, physical means as profound diathermy or procedures with similar effects may increase the efficiency of the therapeutic factor in sites of poor blood circulation

FIG. 8-c and d; Pulmonary metastases, three weeks after therapeutic vaccination. Tumor cells in apoptosis, confirmation by TUNEL staining.

The metastatic dissemination is known in the biology of cancer as being composed in general by tumor cells in more active metabolism than the primary tumor and as a consequence in a more active proliferation. The relevance of this finding for the in situ production of F5 is the great sensitivity to apoptotic factors as it is shown by the histological findings of this figure.

FIG. 8-e, f and g; Apoptosis of VX2 at three degree of magnification.

FIG. 9-a, b, c and d; metastatic spread at various magnifications of VX2 cells in the alveolar walls and in compact agglomerations. The extended apoptosis of tumoral cells is yielded as two weeks after Active Immunization of the tumor-carrier rabbit with FIV agent.

FIG. 10-a, b,c and d; The metastatic tumoral tissue destroyed under the effect of anticancer vaccination is replaced by conjunctive (scar) tissue, demonstrable by Van Gieson staining. The production of conjunctive tissue as a replacement of cancer is not usual and generally the wound in cancer tissue do not show tendency of cicatrisation. The phenomenon is an exception in the evolution of tissular destructions in tumor biology.

FIG. 11-a and b; The Rat Hepatoma presents defined advantages for experimental oncology. The long survival of models grants enough time for immunological processes that otherwise, in most of other experimental tumors on rodents can not be achieved. The figures of this paragraph show the development of hepatoma on living models on 4 months of development and respectively 2 months, without treatment and in good general condition.

FIG. 11-c show the real size of a 4 month tumor after sacrifice of the animal and lifting the skin.

FIG. 12-a and b; pulmonary metastases of hepatoma visible macroscopic, before fixation. The great tolerance of the host for the tumor enables an extended metastatic spread without alteration of the general condition.

FIG. 13-a, b and c; Microscopy of Hepatoma in Rat, at three degree of magnification. No treatment applied. Reference case.

FIG. 14-a, b, c and d (rat 2424) Hepatoma in rat, two month after the clinical appearance. Three weeks after vaccination with Bov FIV Abs softening and reduction in volume of the tumor followed by death. At autopsy; explosive and extended apoptosis of cancer cells. H/E Staining.

FIG. 15-, b; Similar clinical history as in the previous case. Although the specific reaction for apopotosis is TUNEL, the cellular images as obtained in 15-b with H/E staining are quite unmistakable for determining the nature of the cellular destruction.

FIG. 15-a, c and 15-d; confirmation of apoptosis of the same case by TUNEL reaction.

FIG. 16 The recognition of cellular debris as belonging to a treated and destroyed tumor by F5 may be difficult in absence of morphological criteria. Hepatoma has the advantage of enabling after weeks the recognition of treated and destroyed cancer tissue due to the due to the tinctorial affinities for glycogen of some histochemical stainings.

FIG. 16-a shows the standard staining with Mucin-Carmin of Hepatoma in rat, obtained by biopsy, after fixation in Formalin 4% and the usual histological processing.

FIG. 16-b shows the results of the muci-carmin staining from tissue removed from the lips of a wound after partial cancer surgery. Numerous cancer cells, that couldn't be separated from skin were included in the line of suture a month ago.

FIG. 16-c shows what is left from the rests of a tumor which remained in place after extirpation of the major part of the primary tumor. The case has been previously treated by Active Immunization with FIV. The brightness of the newly stained anatomical items has changed in both cases from the colors of reference from FIG. 16-a, however in spite lack of any figurate pattern the recognition of the hepatom origin is still possible

FIG. 17-a, b, c and d; Apoptotic destruction of small Rat Hepatoma; (2112) in the first three weeks by vaccination with FIV Abs. TUNEL staining in various magnifications.

FIG. 18-a, b, c and d; Lung sections of rat with Hepatoma, Active Immunized with FIV Abs Bov. TUNEL staining. Aapoptosis of micro metastases in the alveolar walls.

FIG. 19-a; Abdominal Scar after surgical extirpation of hepatoma tumor, one month after vaccination with FIV Bov. The tumor growth stopped but didn't show signs of regression. The healing of the surgical wound was unusually rapid (3-4 days) in spite lack of sterility. No tumor recidivism in the following three month.

FIG. 19-b and c: Bilobated aspect of the extirpated tumor.

FIG. 20-a: Destruction “en masse” of a big Hepatoma in Rat that followed by 13 days the Active Immunization of the model with Bov FIV Abs immobilized on Polystyrene beads.

FIG. 20-b; Control (size) tumor of the same age.

FIG. 20-c and d; Magnification of FIG. 20-a. Conspicuous differentiation of acini and lobules of the tumor. The tumor is very friable but not hemorrhagic The red color of the tumor is due to intrinsic histological changes. No traces of blood are found at the tumor section

FIG. 20-e. Magnification of Control tumor.

FIG. 20-f; 01, 02, 03, 04, 05, 06, 08, 09 and 10 Apoptosis fulminant of the whole tumor. Microscopic details of the tumor destruction.

FIG. 21; Schematic development of premunition in cancer;

I: The tumor cells (TC) release Autocrine and Merocrine Growth Factors that bind the Cancer Cell Receptors (CCR). A certain part of the product of secretion enters the general circulation as Shed Antigen (ShA) and may be separated as peptides with cholinergic function.

II. A fraction of the general stock of Naïve B Cells (NBC) have receptors attached covalently to Shed Antigens. Another fraction may be coated by a pellicle of ShA by physical adhesion.

III. B cells with receptors for ShA internalize the antigen, react by Plasma Cell transformation and produce antibodies F II-like. Due to the special conditions of the cancer presence the novel formed antibodies are not released but accumulate in hypermaturated Plasma Cells FIG. 2. Imperfect antibodies, otherwise unable to be released may leak in the general circulation through the solutions of continuity of old Plasma Cells membrane.

IV. The small quantities of incomplete antibodies that have leaked in the organism of the cancer carrier induce in competent B cells (second row of the fourth subsection) partial forming of F III Abs and sensitization of new groups of B cells.

FIG. 22; Scheme of production of FV in cancer carrier(s).

Specific of this invention is the massive production F5 antibodies in the organism of the cancer carrier as an effect of the Primary Immunization with the antigen, without necessity of the booster. The importance of this achievement is not only as a general anticipation of a desirable effect but more specifically, of preventing the establishment of a new regimen of tolerance of the guest of cancer to the novel antibodies. Actually, the procedure is an artificial reproduction of the immunologic switch by which the Spontaneous regression of cancer is possible and in very rare cases actually happens.

The FIV Abs introduced as vaccine in the organism of cancer carrier bind also to preformed and retentive Plasma Cells. Due to their similarity to the non-released FII the FIV Abs function in this occurrence as antigens that provide a co-stimulatory signal to the Plasma transformed B cells. The physiological effect of the co-stimulation is the permeation of the cellular membrane and the release “en masse” of antibodies contained in the premunition phase in Plasma Cells. The ‘imperfect’ antibodies act on the side of external FIV as an endogenous antigen in the production and release of F5 in amounts otherwise not possible to be reached by a conventional Active Immunization.

Experimental confirmation of this mechanism is provided by multiplying the antigenic exposed sites by the procedure of immobilization the Fc of FIV Abs on polystyrene beads. The immunologic following is a huge increase in F5 production in the just short period of time of the Primary Immunization. This effect would not be possible in the absence of Memory Cells but by admitting the pre-existence of the sensitivity of the organism in question to the antigens of the vaccination. In the terms of this invention this is defined as the premunition to the autocrine Growth Factors of cancer.

The FIG. 22 shows: FIV vaccine binds to B cells that have previously undergone a (1) Plasmatic transformation (NPC, IPC and HPC). The not-completed FII antibodies act after release on Sensitised B Cells (SBC) and raise (2) F V that binds and blocks the Cancer Cell Receptors (CCR).

The alternative and parallel way is the classical effect of FIV vaccine on B cells, sensitized or not (3) followed by production of F V and occupancy of CCR on Tumor Cells (TC).

### REFERENCES CITED

1. Angeling-Duclos C et al, Commitment of B lymphocytes to a plasma cell fate is associated with Blimp-1 expression in vivo. J. Immunol. 2000 Nov. 15;15;165(10);5462-71
2. Barre C, Verine J L, Regnier J, Enon B, Houssin A, Chaigne P, Soret J Y. Spontaneous regression of regressive pulmonary metastases from kidney cancer. Myth or reality? Apropos of 2 cases. Ann Urol (Paris) 1986;20(4):275-9.
3. Baselga J, Albanell J. Mechanism of action of anti-HER2 monoclonal antibodies. Ann Oncol 2001;12 Suppl 1:S35-41.
4. Beck G, Munno D W, Levy Z, Dissel H M, Van-Minnen: Neurotrophic activities of trk receptors conserved over 600 million years of evolution. J Neurobiol 2004 July:60(1):12-20
5. Bhattacharya-Chatterjee M, Chatterjee S K, Foon K A. Anti-idiotype vaccine against cancer. Immunol Lett 2000 Sep. 15;74(1):51-8.
6. Bhattacharya-Chatterjee M, Chatterjee S K, Foon K A. The anti-idiotype vaccines for immunotherapy. Curr Opin Mol Ther 2001 February; 3(1):63-9.
7. Bhattachary-Chatterjee M, et al. Counterpoint. Cancer vaccines: Single-epitope ant-idotype vaccine versus multiple-epitope antigen vaccine. Cancer Immunol Immunother 2000 June; 49(3):133-41.
8. Bocchia M, Bronte V, Colombo M P, De Vincentiis A, Di Nicola M, Forni G, Lanata L, Lemoli R M, Massaia M, Rondelli D, Zanon P, Tura S. Antitumor vaccination: Where we stand. Haematologica 2000 November; 85(11):1172-206.
9. Bodey B, Bodey B Jr, Siegel S E, Kaiser H E. Failure of cancer vaccines: The significant limitations of this approach to immunotherapy. Anticancer Res 200 July-August; 20(4): 2665-76.
10. Bodey B. Spontaneous regression of neoplasms: New possibilities for immunotherapy. Expert Opin Biol Ther 2002 June; 2(5):459-76.
11. Brizzolara R A. Patterning multiple antibodies on polystyrene. Biosens Bioelectron; 2000 March; 15(1-2):63-8.
12. Brown G V, Beck H P, Molyneux M, Marsh K. Molecular approaches to epidemiology and clinical aspects of malaris. Parasitol Today. 2000 October; 16(10):448-51.
13. Brummendorf T, Lemmon V. Immunoglobulin superfamily receptors: cis-interactions, intracellular adapters abd alternative splicing regulate adhesion. Curr Opin Cell Biol 2001 October; 13(5):611-8:1.
14. Calmanovici S Identification of a cancer humoral factor in human by stimulating the neuromuscular junction of the amphibian injected with human cancer serum. State of Israel Patent 71537/84
15. Calmanovici SMethod of functional assessment of cancer; humoral factors U.S. Pat. No. 4,644,959/87
16. Calmanovici S Procede de detection d'un facteur cancereux dans un liquide corporel Republique Francaise, Patent 85 05142
17. Calmanovici S. Preparation of antigen (s) par electromyographical protein separation. State of Israel, Patent 10 34 94/92
18. Calmanovici S, Mordochovich D, Cohen E, Sherman Y, Livni N. Morphological changes induced in experimental pulmonary metastasis by a novel immunological treatment. 16^{th} Annual Meeting of the Israel Society for Histochemistry and Cytochemistry, 1997 May 22:3
19. Calmanovici S, et al. Non-specific anti-cancer vaccination in rat: Immunohistochemical aspects. 17^{th} Annual Meeting of the Israel Society for Histochemistry and Cytochemistry, 1998 May 10:21
20. Calmanovici S Plasma Cells in Squamous Lung Carcinoma. Relation with premunition. 23th Annual Meeting of the Israel Society for Histochemistry and Cytochemistry, Jun. 1, 2004
21. Calmanovici S Preparation of idiotypes antibodies to Cancer Humoral Factor (CHF) Application for Patent, State of Israel, October 2004
22. Calmanovici S Production of Antigen Determinants of Cancer Humoral Factor by anti-idiotypes antibodies (FIII). Application for Patent, State of Israel, October, 2004,
23. Calmanovici S Production of competitive antibodies (FIV) to wild growth-cholinergic receptors. Application for Patent, State of Israel, October 2004
24. Ciardiello F, Tortora G. A novel approach in the treatment of cancer: targeting the epidermal growth factor receptor. Clin Cancer Res 2001 October; 7(10):2958-70.
25. Costello R T, Gastaut J A, Olive D. Tumor escape from immune surveillance. Arch Immunol Ther Exp (Warsz) 1999;47(2):83-8.
26. Croft B Y, Hoffman J M. NCI-funded small animal imaging programs. National Cancer Institute. Acad Radiol 2001 April; 8(4):372-4.
27. Erp R van, Linders Y E, van Sommeren A P, Gribnau T C. Characterization of monoclonal antibodies physically adsorbed onto polystyrene latex particles. J Immunol Methods. 1992 Aug. 10; 152(2):191-9.
28. Freeman G J, Freedman A S, Segil J M, Lee G, Whitman J F, Nadler L M. B7, a new member of the Ig superfamily with unique expression on activated and neoplastic B cells. J Immunol. 1989 Oct. 15; 143(8):2714-22.
29. Gasparyan V K. Preparation and sensitization of polystyrene latex beads by some antigens and antibodies. Factors affecting sensitivity and specificity of latex agglutination tests. J Immunoassay Immunochem 2002; 23(3):399-406.
30. Geraads A, Carpentier A, Plagne R. Is spontaneous regression of pulmonary metastasis of renal cancer possible? Apropos of a case with review of the literature. Rev Pneumol Clin 1993; 49(5):233-42.
31. Heller M, von der Ohe M, Kleene R, Mohajeri M H, Schachner M. The immunoglobulin-superfamily molecule basigin is a binding protein for oligomannosidic carbohydrates: An anti-idiotypic approach. J Neurochem. 2003 February; 84(3):557-65.
32. Hoffman J M, Croft B Y. Future directions in small animal imaging. Lab Anim (NY) 2001 March; 30(3):32-5.
33. Hoffman J M. Imaging in cancer: A National Cancer Institute “extraordinary opportunity”. Neoplasia 2000 January-April; 2(1-2):5-8.
34. Hommel M. [Physiopathology of symptoms of malaria. Role of cytokines, cytoadherence and premunition]. Presse Med. 1996 Jan. 20; 25(2):70-6. Review.
35. Hoption Cabb S A, van Netten C, Glover D W. Spontaneous regression: A hidden treasure buried in time. Med Hypotheses 2002 February; 58(2):115-9.
36. Jager D, Jager E, Knuth A. Immune responses to tumor antigens: Implications for antigen specific immunotherapy of cancer. J Clin Pathol 2001 September; 54(9):669-74. Review.
37. Jager E, Knuth A. Clinical cancer vaccine trials. Curr Opin Immunol 2002 April; 14(2):178-82.
38. Kleef R, Jonas W B, Knogler W, Stenzinger W. Fever, cancer incidence and spontaneous remissions. Neuroimmuomodulation. 2001; 9(2):55-64.
39. Leonard D S, Hill A D, Kelly L, Dijkstra B, McDermott. E, O'Higgins N J. Anti-human epidermal growth factor receptor 2 monoclonal antibody therapy for breast cancer. Br J Surg 2002 March; 89(3):262-71
40. Lewis J S, Achilefu S, Garbow J R, Laforest R, Welch M J. Small animal imaging. Current technology and perspectives for oncological imaging. Eur J Cancer 2002 November; 38(16):2173-88.
41. Li L, Shaw P E. Autocrine-mediated activation of STAT3 correlates with cell proliferation in breast carcinoma lines. J Biol Chem 2002 May 17;277(20): 17397-405.
42. Luo F, Wei Y, Kan B. [Anti-tumor immune response against mouse melanoma to xenogeneic vaccination] Zhonghua Zhong Liu Za Zhi 2001 March; 23(2):118-21.
43. Lyerly H K, Morse M A, Clay T M. Surrogate markers of effective anti-tumor immunity. Ann Surg Oncol 2001 April; 8(3):190-1.
44. Manoharan M. Oligonucleotide conjugates as potential antisense drugs with improved uptake, biodistribution, targeted delivery, and mechanism of action. Antisense Nucleic Acid Drug Dev 2002 April; 12(2):103-28.
45. McKeage K, Perry C M. Trastuzumab: A review of its use in the treatment of metastatic breast cancer overexpressing HER2. Drugs 2002; 62(1): 209-43.
46. Mendelsohn J. The epidermal growth factor receptor as a target for cancer therapy. Endocr Relat Cancer 2001 March; 8(1):3-9.
47. Mendelson J. Blockade of receptors for growth factors: An anticancer therapy—The fourth annual Joseph H Burchenal American Association of Cancer research, Clinical Research Award Lecture. Clin Cancer Res 2000 March:6(3):747-53.
48. Mistry N, Harrington W, Lasda E, Wagner E J, Garcia-Blanco M A Of urchins and men: evolution of an alternative splicing unit in fibroblast growth factor receptor genes RNA. 2003 February; 9(2):209-17.
49. Mitchell M S, Harel W. Kan-Mitchell J, et al. Active specific immunotherapy of melanoma with allogeneic cell lysates: Rationale, results and possible mechanisms of action. Ann NY Acad Sci 1993;690: 153.
50. Moingeon P. Cancer vaccines. Vaccine 2001 Jan. 8; 19(11-12):1305-26.
51. Monzavi-Karbassi B, Cunto-Amesty G, Luo P, Kieber-Emmons T. Use of surrogate antigens as vaccines against cancer. Hybrid Hybridomics; 2002 April; 21(2):103-9.
52. Moody T W, Hill J M, Jensen R T. VIP as a trophic factor in the CNS and cancer cells. Peptides 2003 January; 24(1):163-77.
53. Neil Barclay A. Ig-like domains: Evolution from simple interaction molecules to sophisticated antigen recognition. Vol. 96, Issue 26, 14672-14674, December 21, 1999.
54. Niakan B. A hypothesis on the biochemistry of spontaneous remissions of cancer: Coupling of oxidative phosphorylation and the remission of cancer. Cancer Biother Radiopharm. 1999 August; 14(4):297-8.
55. Normanno N, Bianco C, De Luca A, Salomon D. The role of EGF-related peptides in tumor growth. Frontiers in Bioscience 6, d685-707, May 1, 2001.
56. Papac R J. Spontaneous regression of cancer: Possible mechanisms. [Review] [100 refs]. In Vivo. 12(6):571-8, 1998 November-December.
57. Paulus M J, Gleason S S, Easterly M E, Foltz C J. A review of high-resolution X-ray computed tomography and other imaging modalities for small animal research. Lab Anim (NY) 2001 March; 30(3):36-45.
58. Pegram M, Slamon D. Biological rationale for HER2/neu (c-erbB2) as a target for monoclonal antibody therapy. Semin Oncol 2000 October; 27 (5 Sppl 9):13-9.
59. Qian W, Yao D, Yu F, Xu B, Zhou R. Bao X, Lu Z. Immobilization of antibodies on ultraflat polystyrene surfaces. Clin Chem. 2000 September; 46(9):1456-63.
60. Renno T, Lebecque S, Renard N, Saeland S, Vicari A. What's new in the field of cancer vaccines? Cell Mol Life Sci. 2003 July; 60(7):1296-310.
61. Rubin I, Yarden Y. The basic biology of HER2. Ann Oncol 2001;12 Suppl 1:S3-8.
62. Sahin A A. Biologic and clinical significance of HER-2/neu (cerbB-2) in breast cancer. Adv Anat Pathol 2000 May; 7 (3):158-66.
63. Sciammas R, Davis M M, Modular nature of Blimp-1 in the regulation of gene expression during B cell maturation; J Immunol. 2004 May 1;172(9);5427-40
64. Seachrist L. Spontaneous cancer remissions spark questions. J Natl Cancer Inst. 1993 Dec. 1;85(23):1892-5.
65. Sethi T, Langdon S, Smyh J, Rozengurt E. Growth of small cell lung cancer cells: Stimulation by multiple neuropeptides and inhibition by broad spectrum antagonists. Cancer res. 1992 May 1;52(9 Suppl.): 2737s-2742s.
66. Shaffer A L and al, XPB1, downstream of Blimp-1 expands the secretory apparatus of other organelles, and increases protein synthesis in plasma cell differentiation Immunity, 2004 July; 21(1)81-93
67. Shaffer A L et al., Blimp-1 orchestrates plasma cell differentiation by extinguishing mature B cell gene expression program. Immunity. 2002 July; 17(1)51-62
68. Shen M, Horbett T A. The effects of surface chemistry and absorbed proteins on monocyte/macrophage adhesion to chemically modified polystyrene surfaces. J Biomed Mater Res. 2001 Dec. 5; 57(3):336-45.
69. Shmanai V V, Nikolayeva T A, Vinokurova L G, Litoshka A A. Oriented antibody immobilization to polystyrene macrocarriers for immunoassay. BMC Biotechnol 2001; 1(1):4.
70. Sioud M. How does autoimmunity cause tumor regression? A potential mechanism involving cross-reaction through epitope mimicry. Mol Med 2002 March; 8(3): 115-9. Review.
71. Smith T, Felger I, Tanner M, Beck H P. Premunition in Plasmodium infection: Insights from the epidemiology of multiple infections. Trans R Soc Trop Med Hyg. 1999 February; 93 Suppl 1:59-64.
72. Soe-Soe, khin-Saw-Aye, Htay-Aung, Nay-Win, Tin-Aung, Than Swe, Roussilhon C, Perignon J L, Druilhe P. Premunition against Plasmodium falciparum in a malaria hyperendemic village in Myanmar Trans R Soc Trop Med Hyg. 2001 January-February; 95(1):81-4.
73. Syvalahti E, Hietala J. [The use of imaging techniques in drug research]. Duodecim 1998; 114(10):1019-26.
74. Taheri M, Saragovi U, Fuks A, Makkerh J, Mort J, Stanners C P. Self recognition in the Ig superfamily. Identification of precise subdomains in carcinoembryonic antigen required for intercellular adhesion. J Biol Chem 2000 September. 1; 275(35):26935-43:1.
75. Tait N J. Spontaneous remission of cancer. N Z Med J. 1993 October. 13; 106(965):435.
76. Tisseuil J. [Is the notion of premonition an obsolete one?] Bull Soc Pathol Exot Filiales. 1971 July-August; 64(4):429-33.
77. Weissleder R. [Experimental radiology].Radiology 2001 February; 41(2):115.
78. Xhallis G B, Stam H J. The spontaneous regression of cancer. A review of cases from 1900 to 1987. Acta Oncol. 1990; 29(5):545-50.

1. A method of generating a therapeutically effective amount of F5 antibodies in a subject in need thereof, the method comprising administering to the subject a sufficient amount of F4 antibodies.

2. The method of claim 1, wherein the subject, human or animal has cancer of any histological type in its primary form or with metastases spread.

3. The method of claim 1, wherein the subject has a solid or liquid tumor.

4. The method of claim 1, wherein said F4 antibodies are co-formulated with polystyrene.

5. A pharmaceutical composition comprising F4 antibodies and polystyrene.
